(19) United States
(12) Patent Application Publication
(10) Pub. No.: US 2013/0028920 A1
(43) Pub. Date: Jan. 31, 2013

(54) STABILIZED ANTIBODY PREPARATIONS AND USES THEREOF

(71) Applicants: Robert Gurny, Geneva (CH); Leonardo Scapozza, Grens (CH); Yvonne Westermaier, Flims (CH); Marieke Veurink, Geneve (CH)

(72) Inventors: Robert Gurny, Geneva (CH); Leonardo Scapozza, Grens (CH); Yvonne Westermaier, Flims (CH); Marieke Veurink, Geneve (CH)

(73) Assignee: UNIVERSITE DE GENEVE, GENEVA 4 (CH)

(21) Appl. No.: 13/637,051

(22) PCT Filed: Mar. 31, 2011
(86) PCT No.: PCT/IB2011/051373

§ 371 Date: Sep. 25, 2012

Related U.S. Application Data
(60) Provisional application No. 61/319,313, filed on Mar. 31, 2010.

(51) Int. Cl. A61K 39/395; A61P 27/02; A61P 9/00; A61P 11/06; A61P 31/14; A61P 1/00

(52) U.S. Cl. 424/182.1

(57) ABSTRACT

The present invention is directed to stabilized intact antibody formulations, related methods and uses thereof. In particular, the invention relates to a method of stabilizing an intact antibody in a liquid carrier.

### FIELD OF THE INVENTION

The present invention relates to antibody preparations, in particular to methods for stabilizing antibodies and antibody preparations, to antibody preparations having increased stability, and to uses thereof. The invention further relates to pharmaceutical compositions comprising a stabilized antibody preparation.

### BACKGROUND OF THE INVENTION

Therapeutic antibodies are currently the fastest growing area of biopharmaceuticals. The recent development of chimeric and fully-humanized monoclonal antibodies has spawned an unprecedented interest in using these molecules as therapeutic agents since they can specifically target molecules implicated in disease, thus essentially side-stepping the secondary effects that may be associated with conventional drug therapies. Recent progress in gene recombinant technology has enabled the large scale production of physiologically active proteins such as monoclonal antibodies for diagnostic and therapeutic applications.

The provision of stable therapeutic protein formulations, in particular stable antibody formulations, presents a challenge. Physical and chemical instability of antibodies in aqueous media is a complex function of solution conditions and temperature. Antibodies are, for example, susceptible to deamidation, isomerization, oxidation, proteolysis, aggregation and other covalent modifications. Degradation of antibody formulations due to aggregation phenomena is a particular problem. Not only does the formation of aggregates lead to a reduction in antibody activity, thereby reducing the efficacy of the protein drug, but may also result in potential clinical side-effects or toxicity since aggregates can increase the immunogenicity of the protein drug (Demeule et al., 2006, Eur. J. Pharm. Biopharm., 62:121-30).

Antibody aggregation is also a source of batch to batch variations in the antibody production chain and its control leads to regulatory and quality control burdens, with their associated costs.

Further, the propensity of antibodies to aggregate adversely affects the stability of therapeutic antibody formulations on storage, including their shelf-life, and their useable administration time once removed from optimal storage conditions.

Unlike other model proteins, antibody stability is not necessarily dependent on protein concentration, buffer concentration, salt concentration, or agitation. Antibody stabilization is problematic since antibodies are very sensitive to environmental conditions which render aggregation and degradation very difficult to predict, notably because each antibody may have a very specific and characteristic stability profile. The lack of effect for primary factors commonly known to affect physical stability suggests that the mechanism(s) of antibody stability is counter-intuitive and may differ from that of other well-studied proteins.

To date, most therapeutic monoclonal antibodies introduced into clinical use are of the antibody type immunoglobulin G (IgG). For example, bevacizumab (Avastin®) is a recombinant monoclonal humanized IgG1 antibody with a molecular weight of 149 kDa that binds to and inhibits the biologic activity of vascular endothelial growth factor (VEGF). VEGF is known to play a pivotal role in tumour angiogenesis and is a significant mitogenic stimulus for arterial, venous and lymphatic endothelial cells. The addition of bevacizumab to chemotherapy has been shown to increase overall response rate, duration of response and survival for patients with metastatic colon cancer. Bevacizumab is beneficial in first line non-small cell lung cancer, metastatic breast cancer and second line metastatic colorectal cancer. Bevacizumab is also beneficial in the treatment of neovascular age-related macular degeneration (AMD), a common form of progressive age-related vision loss.

A number of approaches have been investigated to attempt to improve antibody stability. These include approaches based on the addition of ‘stabilizing’ agents to a solution containing the immunoglobulin, and attempts to engender single amino acid mutations at the site(s) implicated in the formation of aggregates on the immunoglobulin molecules. Examples of species investigated as ‘stabilizing’ agents in prior attempts to improve stability of immunoglobulin in solution include polysorbate-based surfactants (GB 2175906), amino acids (EP 0025275, WO 2005/049078), polyethers (EP 0018609), glycerin, albumin, dextran sulphate (U.S. Pat. No. 4,808,705). The success of this approach has, however, been limited. It is believed that one reason for this limited success is that the ‘stabilizing’ agents are directed at optimizing the environment in which the immunoglobulin is contained, and not specifically at interfering with the mechanism of interaction of immunoglobulin molecules in the formation of aggregates. This approach also has limitations in regard of the quantity of stabilizing agent(s) that may be required to produce a positive effect; such quantities may have other detrimental effects on immunoglobulin molecules such as protein unfolding (e.g. for surfactants), or on the suitability and safety of the ‘stabilized’ preparations for clinical administration.

Single amino acid mutations to immunoglobulins could provide a method of specifically targeting sites implicated in aggregation, but such an approach necessarily modifies the structure of the immunoglobulin, and this may affect both its clinical efficacy, and its immunogenicity in the recipient, which can create undesirable side effects, such as an immune response against the therapeutic agent.

Despite the previous investigations on preventing antibody aggregation, the precise nature of the antibody-antibody binding, and particularly the nature of the antibody-antibody contact surfaces in antibody aggregates, remains unclear.

Although many different approaches have been proposed, and certain methods have been incorporated into antibody formulations, aggregation is still an issue. There is to date no available single, effective and widely applicable solution to the aggregation of immunoglobulins used for clinical applications.

In view of the above, there is an on-going need to provide effective methods for inhibiting and/or reducing aggregation of antibodies.

In order to better address the problems of antibody aggregation, there is a need to identify the key regions of antibodies implicated in aggregation, and to understand the nature of the antibody-antibody contact surfaces in antibody aggregates.

Since aggregation is a major issue for the production, formulation and stability of therapeutic antibodies, and can lead to loss of biological activity, loss of solubility and even increased immunogenicity, particularly there is an on-going need to provide therapeutic antibody preparations, particularly formulations of monoclonal antibodies, which provide improved stability and shelf-life of those antibodies.

### SUMMARY OF THE INVENTION

The invention relates to the unexpected finding of a method of stabilizing an intact antibody, notably decreasing its aggregation propensity, by inhibiting an aggregation contact region of the Fc region, in particular a CH domain of the Fc region of said intact antibody. The invention relates to the further finding that inhibition of the aggregation contact region of said Fc region, in particular a CH domain of the Fc region of said antibody can be achieved by masking at least one specific residue from said region, typically an amino acid sequence comprising this specific amino acid, which is shared by the CH domains of the Fc regions from most of the therapeutic monoclonal antibodies currently commercialized or under development. In particular, the inventors have unexpectedly found that aggregation of intact antibodies may be modulated by blocking, or masking, at least one of the lysine residues corresponding to Lys445B and Lys383B of an IgG1 crystal structure (Protein Data Bank (PDB) identifier “1IGY”, Harris et al., 1998, J. Mol. Biol., Vol. 275, 6, p 861-872) on the Fc region, in particular a CH domain of the Fc region, of the intact antibody molecule, which is implicated in the formation of aggregates. Blocking, or preventing, antibody-antibody interactions involving the said lysine residues prevents, or reverses, aggregation inducing contacts between intact antibody molecules.

According to one aspect of the invention, there is provided a method of stabilizing an intact antibody in a liquid carrier comprising modulating aggregation of said intact antibody by inhibiting an aggregation contact region on the Fc region, in particular a CH domain of the Fc region, of the intact antibody.

According to one aspect of the invention, there is provided a method of stabilizing an intact antibody in a liquid carrier comprising modulating aggregation of said intact antibody by masking or binding a lysine residue in position number 8 (eight) of an amino acid sequence of SEQ ID NO: 2 comprised in the Fc region, in particular in a CH domain of the said Fc region (e.g. in the CH3 domain), of the said intact antibody molecule.

According to one aspect of the invention, there is provided a method of stabilizing an intact antibody in a liquid carrier comprising modulating aggregation of said intact antibody by masking or binding a lysine residue in position number 8 (eight) of an amino acid sequence of SEQ ID NO: 7 comprised in the Fc region, in particular in a CH domain of the said Fc region (e.g. in the CH3 domain), of the said intact antibody molecule.

According to one aspect of the invention, there is provided a method of stabilizing an intact antibody in a liquid carrier comprising modulating aggregation of said intact antibody by masking a lysine residue corresponding to Lys445B on the Fc region, in particular on a CH domain of the Fc region, of the intact antibody molecule involved in antibody-antibody interactions.

According to one aspect of the invention, there is provided a method of stabilizing an intact antibody in a liquid carrier comprising modulating aggregation of said intact antibody by binding a residue corresponding to Lys445B on the Fc region, in particular on a CH domain of the Fc region, of the intact antibody.

According to one aspect of the invention, there is provided a method of stabilizing an intact antibody in a liquid carrier comprising modulating aggregation of said intact antibody by combining the intact antibody with a modulator compound having binding affinity for a Lysine residue selected from the group of lysine residues corresponding to Lys383B and Lys445B on the Fc region of an IgG1 crystal structure (Protein Data Bank (PDB) identifier “1IGY”, Harris et al., 1998, above), in particular on a CH domain of the Fc region, of the intact antibody. According to one aspect of the invention, there is provided a method of stabilizing an intact antibody in a liquid carrier comprising modulating aggregation of said intact antibody by combining the intact antibody with a modulator compound having binding affinity for the lysine residue corresponding to Lys445B on the Fc region, in particular on a CH domain of the Fc region of the intact antibody.

According to one aspect of the invention, there is provided, a stable antibody formulation comprising a liquid carrier, an intact antibody and a modulator compound, said modulator compound having binding affinity for a residue corresponding to Lys445B on the Fc region of the intact antibody.

According to one aspect of the invention, there is provided a stable antibody formulation comprising a liquid carrier, an intact antibody and a modulator compound having binding affinity for a lysine residue selected from the group of lysine residues corresponding to Lys383B and Lys445B on the Fc region, in particular on a CH domain of the Fc region of the intact antibody. According to one aspect of the invention, there is provided a stable antibody formulation comprising a liquid carrier, an intact antibody and a modulator compound which binds a lysine residue corresponding to Lys445B on the Fc region in particular on a CH domain of the Fc region of the intact antibody.

According to one aspect of the invention, there is provided the use of a modulator compound having binding affinity for a lysine residue corresponding to Lys445B on the Fc region of IgG1, for stabilizing a formulation of an intact antibody in a liquid carrier. According to one aspect of the invention, there is provided a stable antibody formulation comprising a liquid carrier, an intact antibody and a compound of the formula (I):

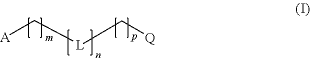

wherein n=0 or 1, m and p are each independently 0 or 1; A is a negatively charged anchor moiety, for example selected from a carboxy, phosphate, phosphonate, phosphinate, phosphorothioate, sulfate, or sulfonate moiety. A may preferably be selected from a phosphonate moiety, a phosphate moiety, or a bioisostere thereof; L is an optional linker group wherein, when present, L is a C1-C₆ alkyl, C₁-C₆ carbonyl, C₁-C₆ ether, optionally substituted by one or more group(s) independently selected from C₁-C₆ alkyl, hydroxy, C₁-C₆ alkoxy, ketone, halo or carboxy group, or a substituted 5- or 6-membered alicyclic, heteroalicyclic, aromatic or heteroaromatic group containing from 0 to 3 heteroatoms selected from a N, O or S, optionally further substituted by one or more group(s) independently selected from a C₁-C₆ alkyl, hydroxy, C₁-C₆ alkoxy, ketone, halo or carboxy group; Q is a cyclic moiety selected from an optionally substitued alicyclic, heteroalicyclic, aromatic or heteroaromatic moiety comprising 1 isolated to 5 five- or six-membered rings, which may be fused, spiro or bridged, and 0 to 5 heteroatoms selected from a N, O or S optionally further substituted by one or more group(s) independently selected from a C₁-C₆ alkyl, hydroxy, C₁-C₆ alkoxy, ketone, aldehyde, carboxy, amine, nitro, thio or halo group, or a pharmaceutically acceptable salt thereof.

According to one aspect of the invention, the compound of formula (I) is selected from a monosaccharide phosphate or a disaccharide phosphate. According to one aspect the compound of formula (I) is a monosaccharide phosphate or a disaccharide phosphate selected from α-D-galactose-1-phosphate, α-lactose-1-phosphate, α-D(+) maltose-1-phosphate and sucrose phosphate, or a pharmaceutically acceptable salt thereof.

According to one aspect of the invention, the compound of formula (I) may be selected from fludarabine, tenofovir, cidofovir, tiludronate or pyridoxal phosphate.

According to one aspect of the invention, the compound of formula (I) may be selected from fludarabine, tenofovir, cidofovir or tiludronate.

According to one aspect the compound of formula (I) is selected from a compound of formula (A):

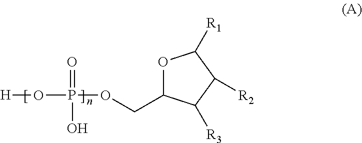

wherein R₁ is a nucleobase; R₂ is H or OR₄ wherein R₄ is H or a C₁₋₄ alkyl group; R₃ is H or OR₅ wherein R₅ is H or a C₁₋₄ alkyl group; and n is an integral from 1-3, or a pharmaceutically acceptable salt thereof. The nucleobase R₁ may be selected from the group consisting of adenine, guanine, thymine, uracil, xanthine, ethanoadenine, inosine, orotidine, or cytosine.

According to one embodiment, the compound of formula (A) is selected from the group comprising adenosine 5′-monophosphate (AMP), adenosine 5′-diphosphate (ADP), or adenosine 5′-triphosphate (ATP). According to one embodiment, the compound of formula (I) is adenosine 5′-monophosphate (AMP).

According to another embodiment, the compound of formula (I) is adenosine 5′-triphosphate (ATP).

According to another embodiment, the compound of formula (I) is adenosine 5′-dihosphate (ADP).

According to another embodiment, the compound of formula (I) is guanosine 5′-monophosphate (GMP).

According to another embodiment, the compound of formula (I) is sucrose phosphate.

The compound of formula (I) may be in the form of its free acid, or may be in the form of a pharmaceutically acceptable salt, for example in the form of a sodium, potassium or calcium salt, e.g. a mono- or di-sodium salt. The invention further encompasses any tautomers of the compounds according to the invention.

It has been unexpectedly found by the inventors that liquid preparations of intact antibodies, in particular intact monoclonal antibodies, may be effectively stabilized by the addition of a compound of formula (I) according to the invention.

Compounds of formula (I) have been shown to exhibit surprisingly favourable intermolecular interaction on systematic docking with the intact monoclonal antibody bevacizumab. Compounds of formula (I) have been shown by computer-assisted systematic docking studies to exhibit preferential binding around the residue corresponding to Lys445B on the Fc region of bevacizumab.

Compounds of the formula (I) according to the invention can reduce the propensity of intact antibodies, such as, for example, the intact monoclonal antibody bevacizumab, to form aggregates in liquid formulations. Compounds of the formula (I) according to the invention can induce the reversion, or breaking, of already formed aggregates of intact antibodies, such as for example bevacizumab, into an essentially monomeric state.

According to another aspect of the invention, there is provided a pharmaceutical formulation such as a formulation formulated for administration to a mammal (e.g. human) comprising a stable antibody formulation according to the invention or a stabilized antibody according to the invention.

According to another aspect of the invention, there is provided a pharmaceutical unit dosage form suitable for administration to a mammal comprising a pharmaceutical formulation according to the invention.

According to another aspect of the invention, there is provided a kit comprising, in one or more container(s), a formulation according to the invention together with instructions of use of said formulation.

According to another aspect of the invention, there is provided a formulation according to the invention for use as a medicament.

In particular embodiments, the medicament may be for use in the treatment or prevention of a disease or disorder selected from immunological diseases, autoimmune diseases, infectious diseases, inflammatory diseases, neurological diseases, neovascular diseases, or oncological diseases.

According to embodiments of the invention, there is provided a formulation according to the invention for the prevention or treatment of a disease or a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD).

According to another aspect of the invention, there is provided a method of stabilizing an intact antibody in aqueous solution.

According to another aspect of the invention, there is provided a process for the preparation of a formulation of an intact antibody in aqueous solution according to the invention.

According to another aspect of the invention, there is provided a stabilized intact antibody or a formulation thereof obtainable by a process or a method according to the invention.

According to another aspect of the invention, there is provided the use of a modulator compound having binding affinity for the residue corresponding to Lys445B on the Fc region of human IgG1, in particular on a CH domain of the Fc region, for stabilizing a formulation of an intact antibody in a liquid carrier.

According to another aspect of the invention, there is provided a method of identifying a modulator compound having activity for modulating intact antibody aggregation comprising:
(i) performing a computer-assisted docking of a candidate compound onto the surface a 3D model of the structure of the said intact antibody;
(ii) identifying a modulator compound that interacts preferentially with the lysine residue in position number 8 of an amino acid sequence having the sequence of SEQ ID NO: 2 comprised in a CH domain of the Fc region of the intact antibody.

According to a further aspect of the invention, there is provided a method of identifying a modulator compound according to the invention wherein the 3D model of the structure of the intact antibody is generated as taught by the present description.

According to another further aspect of the invention, there is provided a method of identifying a modulator compound according to the invention wherein the intact antibody is an antibody listed in Table 1, or which shares a Fc region amino acid sequence with an antibody listed in Table 1. In a particular embodiment, there is provided a method of identifying a modulator compound according to the invention wherein the intact antibody is an antibody which comprises a sequence of SEQ ID NO: 2, in particular which comprises a sequence of SEQ ID NO: 3, 4, 5, 6 or 7 in its Fc region, in particular in a CH domain of its Fc region.

According to another further aspect of the invention, there is provided a method of identifying a modulator compound according to the invention wherein the intact antibody is bevacizumab.

According to another aspect of the invention, there is provided a method of identifying a modulator compound having activity for modulating antibody aggregation comprising: generating a 3D model of the structure of the intact antibody using homology modeling as described in Example 1; performing a computer-assisted docking of a candidate compound onto the surface of the intact antibody bevacizumab; identifying a modulator compound that interacts favourably with a residue corresponding to the Lys445B on the Fc region, in particular on a CH domain of the Fc region of the intact antibody.

According to another aspect of the invention, there is provided a method of identifying a modulator compound having activity for modulating antibody aggregation comprising: generating a 3D model of the structure of the intact antibody using homology modeling as described in Example 1; performing a computer-assisted docking of a candidate compound onto the surface of the intact antibody; identifying a modulator compound that interacts favourably with the lysine residue located in position number 8 of an amino acid sequence having the sequence of SEQ ID NO: 2 comprised in a CH domain of the Fc region of the intact antibody. In a particular embodiment, is provided a method of identifying a modulator according to the invention wherein the modulator compound interacts favourably with the lysine residue located in position number 8 of an amino acid sequence having the sequence of SEQ ID NO: 3 comprised in a CH domain of the Fc region of the intact antibody. In another particular embodiment, is provided a method of identifying a modulator according to the invention wherein the modulator compound interacts favourably with the lysine residue located in position number 28 of an amino acid sequence having the sequence of SEQ ID NO: 4 comprised in a CH domain of the Fc region of the intact antibody. In another particular embodiment, is provided a method of identifying a modulator according to the invention wherein the modulator compound interacts favourably with the lysine residue located in position number 75 of an amino acid sequence having the sequence of SEQ ID NO: 1 comprised in a CH domain of the Fc region of the intact antibody.

According to another aspect of the invention, there is provided the use of a compound identified according to the method of the invention, for stabilizing a formulation of an intact antibody in a liquid carrier.

According to another aspect of the invention, there is provided a method of preventing, treating or ameliorating a disease or a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD), said method comprising administering in a subject in need thereof a prophylactic or therapeutically effective amount of a formulation according to the invention or of a stabilized intact antibody according to the invention.

According to another aspect of the invention, there is provided a use of a formulation according to the invention or of a stabilized intact antibody according to the invention for the preparation of a pharmaceutical formulation for the prevention and/or treatment of a disorder selected from a cancer, rheumatoid arthritis, transplant rejection, blood coagulation, infection with respiratory syncitial virus (RSV), Crohn's disease, cardiovascular disease, auto-immune disease, asthma, paroxysmal nocturnal hemoglobulinuria, psoriasis, or a neovascular age-related macular degeneration disease (AMD).

According to another aspect of the invention, there is provided a use of a formulation according to the invention or of a stabilized intact antibody according to the invention for inhibiting aggregation in the culture, preparation, purification and processing of antibodies prior to formulation into therapeutic preparations.

Other objects and advantages of the present invention will be apparent from the claims and the following detailed description, examples and accompanying drawings, wherein:

FIG. 1 shows the 3D model structure of the intact monoclonal antibody bevacizumab.

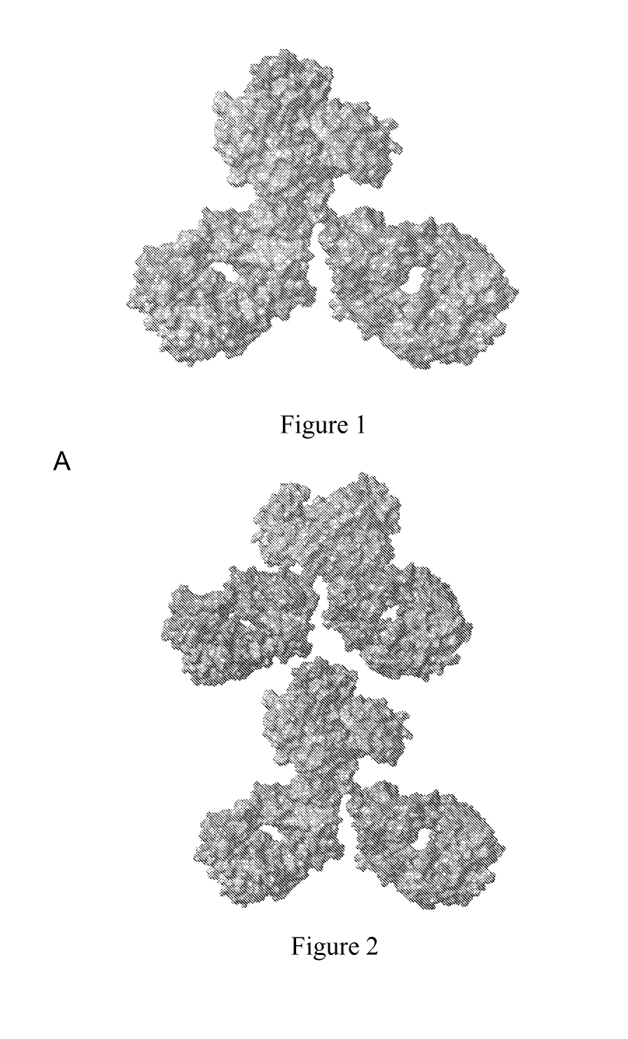

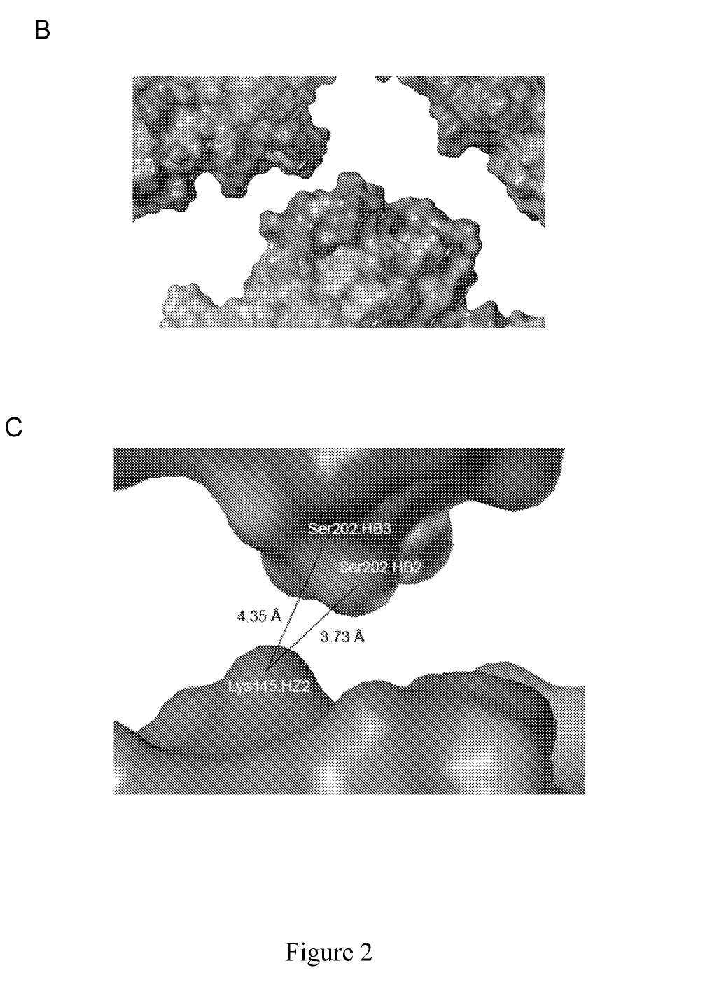

FIG. 2A depicts the aggregation pattern of two bevacizumab antibodies according to the 3D model structure of the intact monoclonal antibody bevacizumab and its symmetry related molecule built using the crystal symmetry of the template IgG1, showing the contact region.

FIG. 2B shows a zoomed image of the antibody-antibody aggregation contact region of two bevacizumab antibodies, depicted in FIG. 2A.

FIG. 2C shows a further zoomed image of the antibody-antibody aggregation contact region shown in FIG. 2B.

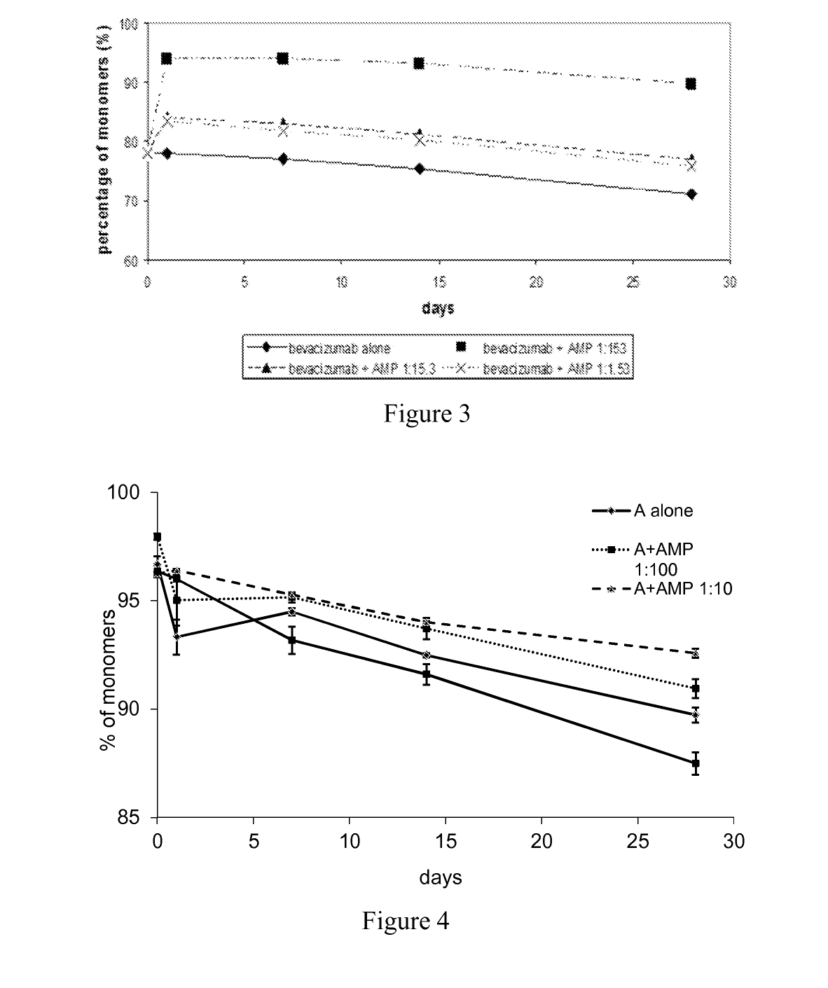

FIG. 3 illustrates the stabilizing effect of a compound of formula (I) on the monoclonal antibody bevacizumab formulated in an aqueous carrier, according to one embodiment of the invention, as described in Example 1.

FIG. 4 is a graphical representation of the stabilizing effect of the compound adenosine 5′-monophosphate on a monoclonal antibody bevacizumab formulated in an unmodified commercial formulation (Avastin® “A”) at different molar ratios as described in Example 2.

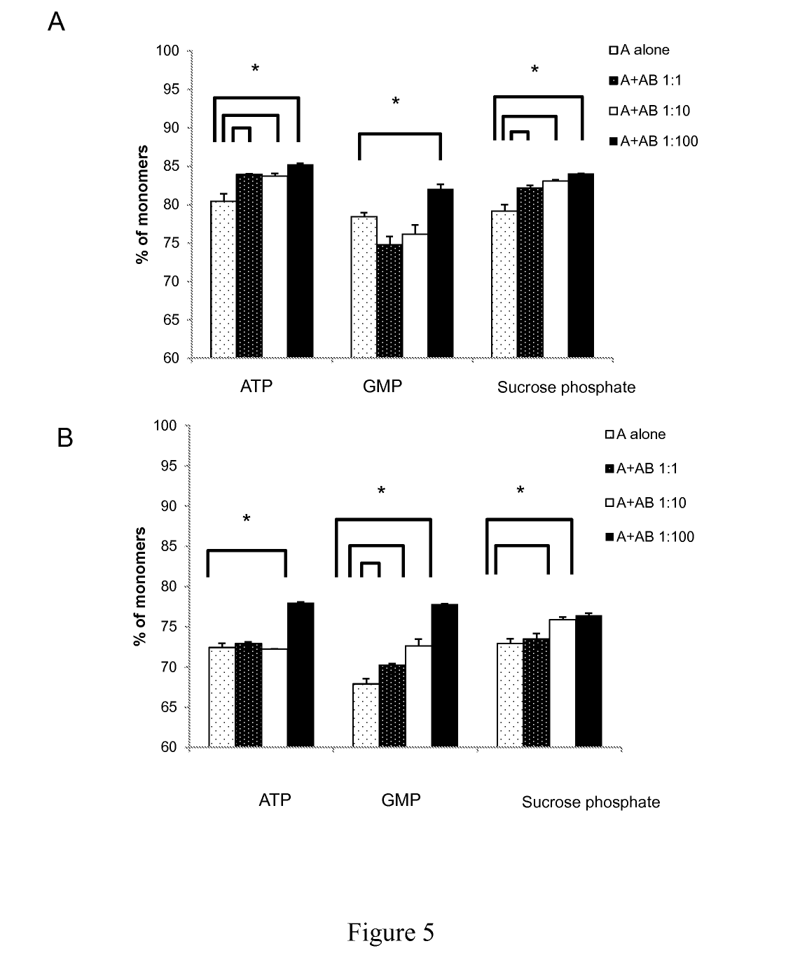

FIG. 5 represents an Avastin® “A” stability comparison in presence and absence of a compound of formula (I) (ATP or GMP or sucrose phosphate “AB”) after storage at 40° C. as described in Example 3. A: after 1 day of storage (t₁); B: After 28 days of storage (t₂₈). The percentage of monomers is presented as mean (n=3)±SD. A significant increase in monomers for a combined formulation compared to Avastin® alone is represented by a *, and is statistically significant (p<0.05).

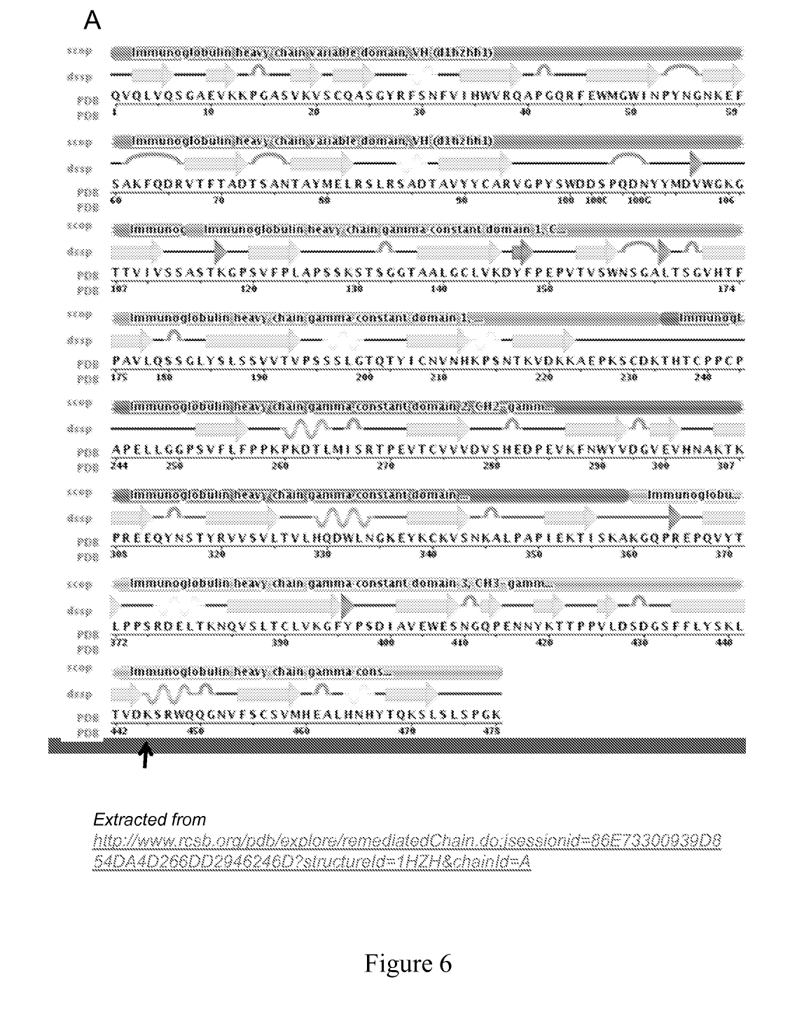

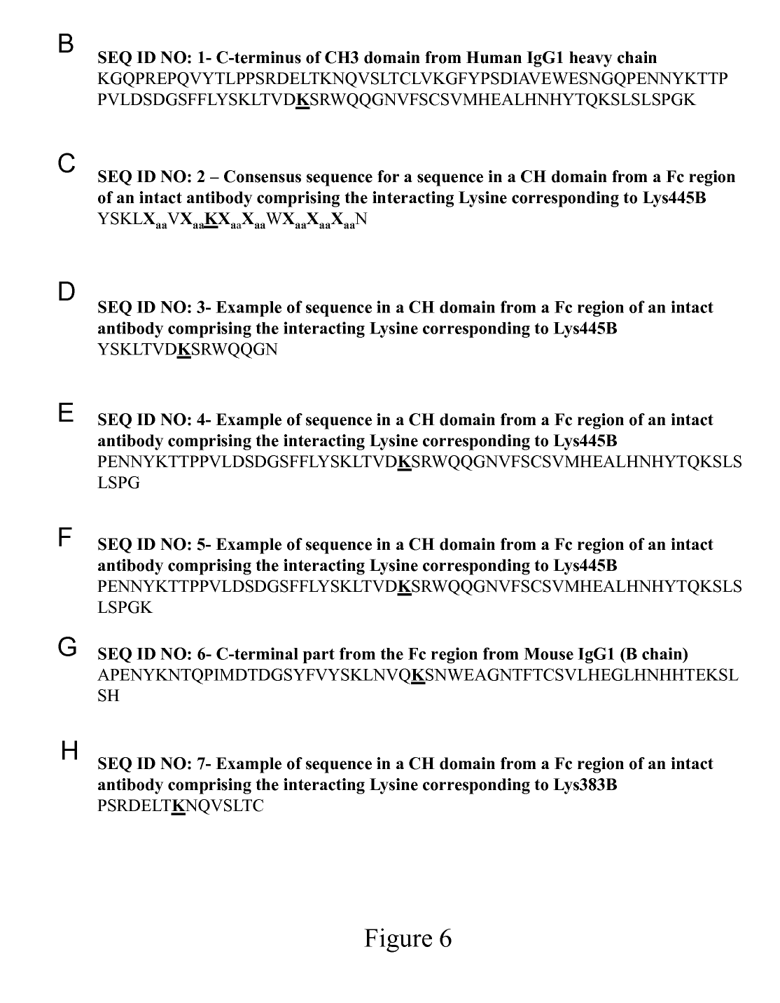

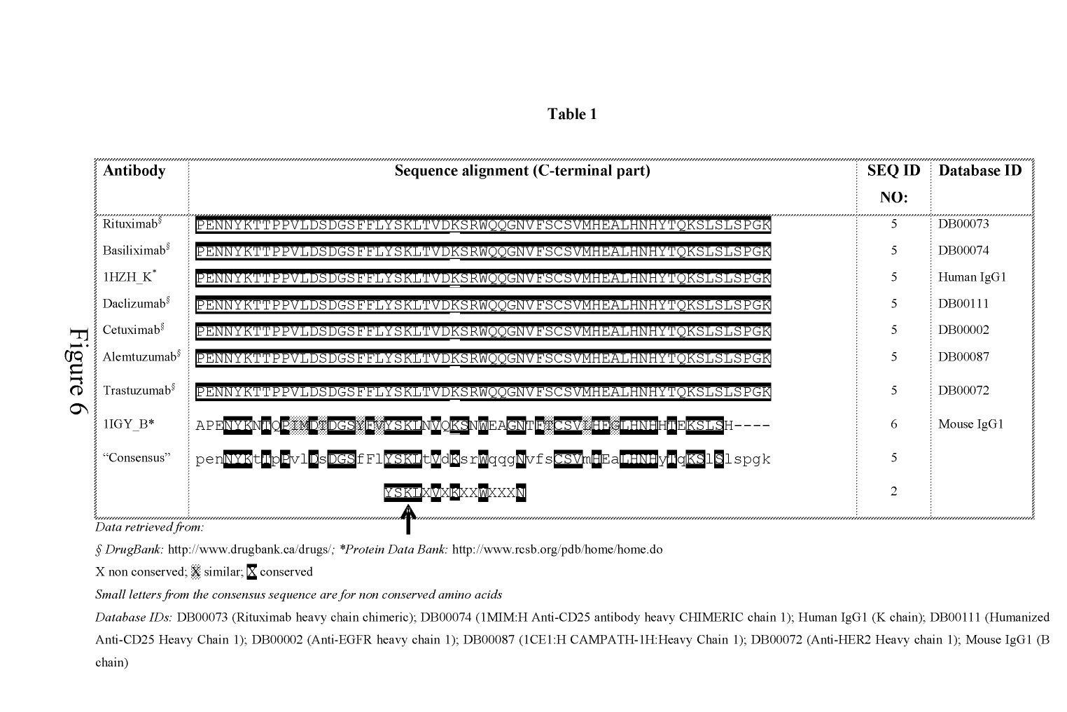

FIG. 6 represents sequences listed in the description and their corresponding SEQ ID NOs. A: Human IgG1 heavy chain; The arrow shows the lysine corresponding to Lys445 in the CH3 domain of the Fc region. (Scop refers to Structural Classification Of Proteins. Dssp refers to an algorithm for assigning secondary structure to proteins described by Kabsch et al., 1983, Dictionary of protein secondary structure: pattern recognition of hydrogen-bonded and geometrical features. Biopolymers, 22 (12), 2577-2367. PDB refers to Protein DataBase. B to H: Amino acid sequences comprised in a CH domain, in particular the CH3 domain of the Fc region of intact antibodies according to the invention comprising the lysine residue involved in antibody-antibody interactions; Xaa refers to an amino acid which can be any amino acid (unspecified amino acid); I: ClustalW multiple amino acid sequence alignments of the C-terminal parts from the Fc regions of IgG that have been crystallized or of commercially available intact monoclonal antibody drugs as compared to a consensus sequence of 61 amino acids of SEQ ID NO: 5 and a consensus sequence of 15 amino acids SEQ ID NO: 2, comprising the interacting lysine residue (arrow).

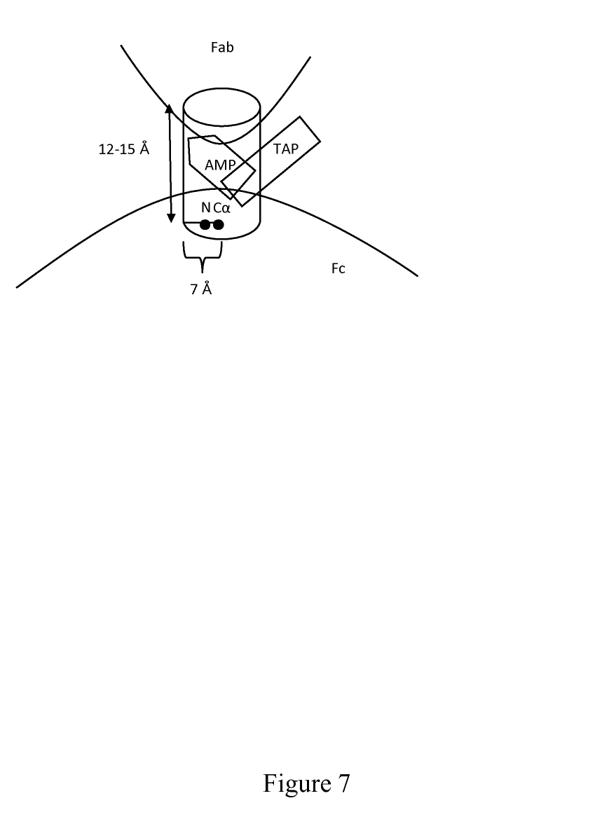

FIG. 7 is a schematic representation of the aggregation model used in a method according to the invention for identifying a modulator of intact antibody aggregation.

### DETAILED DESCRIPTION OF THE INVENTION

The term “intact antibody”, as used herein, refers to antibodies which possess both Fab and Fc regions, as opposed to antibody fragments, e.g. Fab, Fab1 or Fab2 fragments, or single chains thereof. Intact antibodies according to the invention present an aggregation propensity. In a particular embodiment, intact antibodies according to the invention are humanized monoclonal antibodies with specificity for a defined clinical therapeutic target. In particular, intact antibodies according to the invention are monoclonal antibodies comprising an amino acid sequence of SEQ ID NO: 2 within a CH domain of their Fc region, in particular within the CH3 domain of their Fc region. In a further particular embodiment, intact antibodies according to the invention are monoclonal antibodies comprising an amino acid sequence of SEQ ID NO: 3 within a CH domain of their Fc region, in particular within the CH3 domain of their Fc region. In a further particular embodiment, intact antibodies according to the invention are monoclonal antibodies comprising an amino acid sequence of SEQ ID NOs: 4 or 5 within a CH domain of their Fc region, in particular within the CH3 domain of their Fc region. In a further particular embodiment, intact antibodies according to the invention are monoclonal antibodies comprising an amino acid sequence of SEQ ID NO: 1 within a CH domain of their Fc region, in particular within the CH3 domain of their Fc region. In a further particular embodiment, intact antibodies according to the invention are monoclonal antibodies comprising an amino acid sequence of SEQ ID NO: 7 within a CH domain of their Fc region, in particular within the CH3 domain of their Fc region. The term “monoclonal antibody”, as used herein, refers to a preparation of antibody molecules derived from a single clone of antibody producing cells of a uniform amino acid composition. A monoclonal antibody typically exhibits a binding specificity and affinity for a single epitope. Methods for the preparation of monoclonal antibodies are well-known in the art, and are widely based on hybridoma cell production techniques or recombinant antibody engineering techniques.

The term “CH domain of the Fc region” of an intact antibody according to the invention comprises a CH domain of the Fc region derived from immunoglobulins, e.g. IgDs, IgEs and IgGs, such as IgG1, IgG2, IgG2b, IgG3 or IgG4. In a particular embodiment, the CH domain of the Fc region is a CH3 domain of the Fc region of human IgG1 comprising an amino acid sequence of SEQ ID NO: 1.

The amino acid residue designation is taken from an IgG sequence utilized for the modeling of antibody-antibody interactions (PDB identity 1IGY). The interacting lysine according to the invention is in position number 445 on the heavy chains designated B and D, i.e “Lys445B” on the B chain. This residue falls in a highly conserved CH domain of the antibody Fc region, e.g. 33 amino acids from the C-terminal of the human IgG1 heavy chain. However, its numerical position within other full immunoglobulin heavy chains may fluctuate due to natural or engineered variations in the VH (variable) domain closer to the N-terminal, or as a result of the numbering designations of other crystal structures. For this reason, this important lysine residue is referred to as the “lysine residue corresponding to Lys445B” throughout this patent application. By analogy, the expression “Lys383B” is used for an interacting lysine which according to the invention is in position number 383 on the heavy chain designated B.

In embodiments of the invention, the intact antibody can be a full immunoglobulin molecule, particularly monomeric immunoglobulins, e.g. IgDs, IgEs and IgGs, such as IgG1, IgG2, IgG2b, IgG3 or IgG4.

In embodiments of the invention, the intact antibody can be a native antibody.

In other embodiments of the invention, the intact antibody can be an intact monoclonal antibody conjugated to an accessory molecule, also referred to herein as a “conjugated antibody”.

The term “accessory molecule” includes a molecule or an assembly of molecules, of natural or synthetic origin, attached or conjugated to the antibody molecule, providing additional therapeutic, diagnostic, analytical capability or imaging functionality, whereby such functionality is targeted, delivered or activated by the specificity of the antibody.

The accessory molecule may be, for example, an agent active for the treatment of cancer, such as a chemotherapeutic agent, or a radioactive agent.

In embodiments of the invention, the intact antibody can be selected from known therapeutic, diagnostic or preventative intact monoclonal antibody drugs. For example, IgG-based intact antibodies, such as Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab or Ustekinumab may be mentioned.

In a particular embodiment, an intact antibody according to the invention is bevacizumab, notably Avastin® such as described in Presta et al., Cancer Res., 57 (1997), 4593-4599.

The term “alicyclic”, when used alone or in combination with other terms, includes cyclic and polycyclic aliphatic hydrocarbons and bridged cycloalkyl compounds, which may be optionally substituted with one or more functional group(s). Accordingly, the term “alicyclic” includes, but is not limited to, cycloalkyl, cycloalkenyl and cyclalkynyl moieties. This term is exemplified by groups such as cyclopentyl, CH₂-cyclopentyl, cyclohexyl, —CH2-cyclohexyl, cyclohexenylethyl, cyclohexanylethyl and the like, which may optionally be substituted with one or more functional group(s). In polycyclic hydrocarbons, rings may be fused, spiro or bridged.

The term “aliphatic” when used alone or in combination with other terms, comprises both saturated and unsaturated, straight chain or branched hydrocarbons, which may optionally be substituted with one or more functional group(s). Accordingly, the term “aliphatic” includes, but is not limited to, alkyl, alkenyl or alkynyl moieties. This term is exemplified by groups such as methyl, ethyl, n-propyl, i-propyl, n-butyl, s-butyl, i-butyl, t-butyl, n-pentyl, s-pentyl, i-pentyl, t-pentyl, n-hexyl, s-hexyl, ethenyl, propenyl, butenyl, 1-methyl-butene-1-yl, ethynyl, 1-proynyl and the like.

The term “alkyl” when used alone or in combination with other terms, comprises a straight chain or branched C₁-C₆ alkyl which refers to monovalent alkyl groups having 1 to 6 carbon atoms. This term is exemplified by groups such as methyl, ethyl, n-propyl, i-propyl, n-butyl, s-butyl, i-butyl, t-butyl and the like.

The term “alkoxy” when used alone or in combination with other terms, refers to an alkyl group, as previously described, which is attached to the parent molecule through an oxygen atom. This term is exemplified by groups such as methoxy, ethoxy, propoxy, isopropoxy, n-butoxy, t-butoxy, pentoxy, n-hexoxy and the like.

The terms “aromatic” or “aromatic moiety”, when used alone or in combination with other terms, refer to substituted or unsubstituted stable mono- or polycylic hydrocarbon moieties, having preferably 3-18 carbon atoms, preferably 3-10 carbon atoms, comprising at least one ring satisfying the Huckel rule for aromatics. In polycyclic aromatics, rings may be fused, spiro, or bridged.

The terms “heteroalicyclic” or “heterocyclic”, when used alone or in combination with other terms, refer to saturated and unsaturated mono- or polycyclic aliphatic hydrocarbons in which one or more carbon atom(s) in the ring have been replaced with a heteroatom, which may be optionally substituted with one or more functional group(s). In some embodiments the one or more heteroatom(s) are independently N, O or S. This term is exemplified by groups such as pyrrolidinyl, pyrazolidinyl, imidazolinyl, piperidinyl, oxazolidinyl, morpholinyl, thiazolidinyl, tetrahydrofuryl and the like.

The term “heteroaliphatic” when used alone or in combination with other terms, refers to aliphatic moieties (as previously described) in which one or more carbon atom(s) in the ring are replaced with a heteroatom, which may be optionally substituted with one or more functional group(s). The one or more heteroatom(s) may be independently N, O, S, P or Si.

The terms “heteroaromatic” or “heteroaromatic moiety” when used alone or in combination with other terms, refer to stable substituted or unsubstituted aromatic moieties (as previously described), in which one or more carbon atom(s) in the ring have been replaced with a heteroatom. In preferred embodiments, the one or more heteroatom(s) is or are independently N, O or S. This term is exemplified by groups such as pyridyl, pyrimidinyl, pyrazolyl, imidazolyl, thiazolyl, oxazolyl, thiophenyl, furanyl, quinolinyl, dihydroquinazoyl and the like.

Unless otherwise constrained by the definition of the individual substituent, the term “substituted” refers to groups substituted with from 1 to 5 substituents selected from the group consisting of amino, halo, hydroxyl, C₁-C₆ alkoxy, optionally substituted C₁-C₆ alkyl groups such as hydroxyl C₁-C₆ alkyl (e.g. hydroxylmethyl) and the like.

According to a particular embodiment, optionally substituted Q groups are Q groups optionally substituted by hydroxyl, C₁-C₆ alkoxy, or C₁-C₆ alkyl groups such as hydroxyl C₁-C₆ alkyl (e.g. hydroxylmethyl) and the like.

The term “binding affinity” relates to a propensity to interact with, or bind to site(s) within a CH domain of the Fc region of intact antibody molecules that are implicated in antibody-antibody contacts and in the initiation of antibody-antibody aggregation. Typically, the binding affinity may be estimated by modelling using docking scoring according to a method as taught by the present invention.

The term “age-related macular degeneration” (AMD) includes an eye progressive disease presenting an onset usually after age 60 that progressively destroys the macula, the central portion of the retina, impairing central vision.

The term “cancer” includes metastatic and non-metastatic cancers such as colon cancer, rectal cancer, breast cancer, renal cell carcinoma, glioblastoma multiforme, lung cancer, ovarian cancer, prostate cancer, liver cancer, pancreatic cancer, bone cancer, bone metastasis, leukemias, brain cancers, testicular cancer, uterine cancers, cervical cancers, endometrial cancer or other cancers responsive to monoclonal antibody-based therapy.

The term “effective amount” as used herein refers to an amount of at least one polypeptide or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

The term “efficacy” of a treatment according to the invention can be measured based on changes in the course of a disease in response to a use or a method according to the invention. For example, the efficacy of a treatment of a cancer according to the invention can be measured by a reduction of tumor volume, and/or an increase of progression free survival time.

The term “pharmaceutical formulation” refers to preparations which are in such a form as to permit biological activity of the active ingredient(s) to be unequivocally effective and which contain no additional component(s) which would be toxic to subjects to which the said formulation would be administered.

The term “pharmaceutically acceptable salt” refers to a salt that retains the desired activity of the defined compound (i.e. compound of formula (I)) and does not cause any undesired toxicological effects. According to certain embodiments of the invention, the pharmaceutically acceptable salt may be a basic addition salt, such as a sodium, potassium, magnesium or calcium salt. A preferred pharmaceutically acceptable salt of a compound of formula (I) is a sodium salt, e.g. a mono- or di-sodium salt.

The term “stable” or “stabilized” refers in the context of the invention to formulations in which the antibody therein retains its physical stability (e.g. level of aggregation or aggregation propensity decreased, absence of precipitation or denaturation) and/or chemical stability (e.g. absence of chemically altered forms) upon storage or processing. Stability of the antibody formulations according to the invention may be measured by various techniques known to the skilled person in the art. For example, stability can be measured by aggregation state measurements (e.g. by Multi-Angle Light Scattering (MALS) after separation by Asymmetrical Flow Field-Flow Fractionation (AFFF), high performance size exclusion chromatography, analytical ultracentrifugation, fluorescence microscopy or electron microscopy). Preferably, the stability of the formulation is measured at a selected temperature and/or for a selected storage time. Typically, the stability of a formulation according to the invention is measured at a temperature of 40° C. for a period of 35 days.

According to a particular embodiment, the stability of a formulation according to the invention is measured at a temperature of 40° C. for a period of at least 28 days.

The term “subject” as used herein refers to mammals. For example, mammals contemplated by the present invention include humans, primates, domesticated animals such as cattle, sheep, pigs, horses, laboratory rodents and the like.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly in humans, and includes: (a) preventing the disease from occurring in a subject which may be predisposed to the disease, but has not yet been diagnosed as having it, such as a preventive early asymptomatic intervention; (b) inhibiting the disease, i.e., arresting its development; or relieving the disease, i.e., causing regression of the disease and/or its symptoms or conditions such as the improvement or remediation of damage. In particular, the methods, uses, formulations and compositions according to the invention are useful in the preservation of vision and/or prevention of vision loss in patients with age-related macular degeneration and/or in the treatment of cancers.

According to one aspect of the invention, there is provided a method of stabilizing an intact antibody in a liquid carrier comprising modulating aggregation of said intact antibody by inhibiting an aggregation contact region on the Fc region, in particular a CH domain of the Fc region, of the intact antibody. In a particular embodiment, there is provided a method according to the invention wherein the CH domain is a CH3 domain of the human IgG heavy chain. In a further particular embodiment, the CH domain is a CH3 domain of the human IgG1 heavy chain as defined in Saphire et al., 2001, Science, 293:1155-9 and is of SEQ ID NO: 1.

According to one aspect of the invention, there is provided a method of stabilizing an intact antibody in a liquid carrier comprising modulating aggregation of said intact antibody by masking or binding a lysine residue located in position number 8 of an amino acid sequence of SEQ ID NO: 2 comprised in the Fc region, in particular in a CH domain of the said Fc region (e.g. in the CH3 domain), of the said intact antibody molecule. In a particular embodiment, there is provided a method of stabilizing an intact antibody according to the invention by masking or binding to a lysine residue located in position number 8 from an amino acid sequence of SEQ ID NO: 3 comprised in the Fc region, in particular in a CH domain of the said Fc region (e.g. in the CH3 domain), of the said intact antibody molecule. In another particular embodiment, there is provided a method of stabilizing an intact antibody according to the invention by masking or binding to a Lysine residue located in position number 28 of an amino acid sequence of SEQ ID NOs: 4 or 5 comprised in the Fc region, in particular in a CH domain of the said Fc region (e.g. in the CH3 domain), of the said intact antibody molecule. In another particular embodiment, there is provided a method of stabilizing an intact antibody according to the invention by masking or binding to a lysine residue located in position number 8 from an amino acid sequence of SEQ ID NO: 7 comprised in the Fc region, in particular in a CH domain of the said Fc region (e.g. in the CH3 domain), of the said intact antibody molecule.

According to one aspect of the invention, there is provided a method of stabilizing an intact antibody in a liquid carrier comprising modulating aggregation of said intact antibody by masking the residue corresponding to Lys445B on the Fc region, in particular a CH domain of the Fc region, of the intact antibody (as defined in Harris et al., 1998, above). In a particular embodiment, there is provided a method according to the invention wherein the Lysine residue located in position number 75 of SEQ ID NO: 1 is masked (this lysine residue corresponding to the Lysine in position 445 of the sequence of the full length heavy chain of human IgG1 as defined in Saphire et al. 2001, above.

According to one aspect of the invention, there is provided a method of stabilizing an intact antibody in a liquid carrier comprising modulating aggregation of said intact antibody by combining the intact antibody with a modulator compound having binding affinity for a lysine residue selected from the group of lysine residues corresponding to Lys383B and Lys445B on the Fc region, in particular a CH domain of the Fc region of the intact antibody (as defined in Harris et al., 1998, above). According to one aspect of the invention, there is provided a method of stabilizing an intact antibody in a liquid carrier comprising modulating aggregation of said intact antibody by combining the intact antibody with a modulator compound having binding affinity for a lysine residue corresponding to Lys445B on the Fc region, in particular a CH domain of the Fc region, of the intact antibody.

The inventors have for the first time provided a 3D model of the intact monoclonal antibody bevacizumab, and have for the first time successfully provided the 3D aggregation model for the intact monoclonal antibody bevacizumab. Advantageously, based on these 3D structural models, the inventors have made it possible to provide a better understanding of the antibody-antibody contact surface in antibody aggregation. Bevacizumab is an intact humanized monoclonal IgG1 antibody formed by a Fab region responsible for its activity and a Fc region derived from IgG1. The Fc region of IgG1 is conserved in bevacizumab. The 3D structure of bevacizumab was elucidated by the inventors by computer-assisted modelling techniques based on the crystal structures of the two Fab regions of bevacizumab and the full immunoglobulin antibody IgG1. FIG. 1 depicts the surface of the 3Dstructure of bevacizumab.

The inventors have for the first time successfully elucidated the 3D aggregation model of the intact monoclonal antibody bevacizumab. The 3D aggregation model of bevacizumab was elucidated by the inventors using computer-assisted modelling techniques taking into account the homology between bevacizumab and IgG1, the crystal structure of the Fabs of bevacizumab, the known crystal symmetry of the full immunoglobulin IgG1, and the 3D model of the structure of bevacizumab, according to the procedure detailed in Example 1. The 3D aggregation model of bevacizumab obtained by the inventors is shown in FIG. 2A.

The inventors have unexpectedly found that a single antibody-antibody contact zone is key to the formation of an aggregation-inducing contact between antibody molecules, based on their novel 3D aggregation model of bevacizumab. Moreover, the inventors have unexpectedly found that aggregation of intact monoclonal antibodies may be modulated by binding to, or masking, a specific lysine residue, corresponding to Lys445B (Harris et al., 1998, above) in the Fc region of the intact antibody molecule, thereby blocking aggregation inducing antibody-antibody interaction(s) involving the residue corresponding to Lys445B.

Support for the involvement of a lysine residue corresponding to Lys445B on a CH domain of the Fc region in the antibody aggregation mechanism is provided by the 3D aggregation model of bevacizumab, which shows that the only close crystal contact between two antibodies is represented by the interaction of the serine residue Ser202 of chain A, belonging to one Fab arm of a first bevacizumab, and the lysine residue Lys445 of chain B, which is part of the Fc of the second bevacizumab. The two least atom-atom distances were measured as HB2 (Ser202) to HZ2 (Lys445)=3.73 Å and HB3 (Ser202) to HZ2 (Lys445)=4.35 Å, i.e. sufficiently close for binding between the two antibody molecules to occur. Support for the modulation of aggregation of intact monoclonal antibodies by blocking, or preventing, antibody interaction with the residue corresponding to Lys445B is also provided by computer-assisted docking studies, coupled with experimental stability studies on bevacizumab formulations in liquid carrier. From these studies, it is seen that compounds which are effective in modulating aggregation of bevacizumab in aqueous bevacizumab formulations all show a favourable interaction pattern with the residue corresponding to Lys445B in computer-assisted docking models, and all show a most favorable interaction pattern with the residue corresponding to Lys445B when compared to the other residues on the surface of bevacizumab (see examples).

The residue corresponding to Lys445B of the Fc region of IgG1 is generally conserved in the Fc region of engineered monoclonal antibodies. Particularly, the residue corresponding to Lys445B of the Fc region of IgG1 is conserved in the Fc region of therapeutic monoclonal antibodies derived from IgG1, such as bevacizumab as shown on Table 1 on FIG. 6F. Accordingly, since this lysine residue corresponding to Lys445B is conserved in the Fc region of therapeutic monoclonal antibodies, it is believed that blocking antibody-antibody interaction involving the lysine residue corresponding to Lys445B is key in inhibiting the aggregation of intact monoclonal antibodies, at a general level. Further, it follows that blocking, or preventing, antibody interaction with a lysine residue located in position 8 of an amino acid sequence having the sequence of SEQ ID NO: 2 comprised in a CH domain of the Fc region of an intact antibody would result in decreasing aggregation propensity of said intact monoclonal antibodies. In particular, blocking, or preventing, antibody interaction with a lysine residue located in position 8 of an amino acid sequence having the sequence of SEQ ID NO: 3 comprised in a CH domain of the Fc region of an intact antibody would be beneficial. In particular, blocking, or preventing, antibody interaction with a lysine residue located in position 28 of an amino acid sequence having the sequence of SEQ ID NOs: 4 or 5 comprised in a CH domain of the Fc region of an intact antibody would be beneficial.

Interaction with the mentioned lysine residue may be provided by a negatively charged moiety on the modulator compound, for example a phosphate, phosphonate, carboxyl, or nitro group. Phosphate or phosphonate groups, having two negative charges, may be preferred. According to a preferred embodiment, the modulator compound terminates in a phosphate or phosphonate group. The phosphate group may be a mono-, di-, or tri-phosphate group. Mono- or di-phosphates may be preferred.

In order to effectively inhibit aggregation of the intact antibody, the modulator compound, when bound to the Fc region of one antibody molecule, should protrude from the surface of the antibody sufficiently to inhibit interaction of a Fab region of a second antibody molecule with the aggregation contact region proximate to the lysine residue corresponding to Lys445B on the Fc region of the first intact antibody molecule. In view of the minimum calculated distances between antibody molecules based on the bevacizumab aggregation model, the modulator compound may suitably be of a size in the range of from about 4 Å to about 30 Å, preferably from about 4 Å to about 20 Å, e.g. from about 8 Å to about 16 Å, such as from about 8 Å to about 13 Å.

Dimensions of molecules of a modulator compound may be inferred by known methods from 3D structures, e.g. based on experimental X-ray or NMR data analysis of a crystal structure, or based on computer generated 3D models (homology models).

According to one aspect, specific compounds having activity for modulating antibody aggregation were selected with the assistance of computer-based molecular interaction models, based on small-molecule interactions with the 3D structure of bevacizumab. Systematic docking of molecules from a library of compounds was performed all-over the intact antibody surface. Intermolecular interactions were assessed with the FlexX score of FlexX 3.1.3™, however other programs permitting the evaluation of molecular interaction strengths may be contemplated. Evaluation was carried out by analysis of the antibody-small molecule interaction scores, the localization of a most favourable antibody-small molecule interaction pattern for a given small molecule on the antibody surface, and visual analysis of all docking poses. Compounds were selected based on the number of docking poses successful in interfering with the antibody-antibody interaction surface.

According to one aspect, the inventors have provided a method of identifying a compound having activity for modulating aggregation of intact antibodies from a library of compounds. According to one aspect there is provided a method of identifying a modulator compound having activity for modulating antibody aggregation comprising: generating a 3D model of the structure of the intact antibody bevacizumab as defined in Example 1; performing a computer-assisted docking of a candidate compound onto the surface of the intact antibody bevacizumab; and identifying a modulator compound that interacts preferentially, for example by using an interfering volume as described in Example 2, with a residue corresponding to Lys445B on the Fc region, in particular on a CH3 domain of the Fc region of the intact antibody bevacizumab.

Optionally, a compound that has been identified as a compound which interacts preferentially with a residue corresponding to Lys445B on the surface of the intact antibody bevacizumab, may be visually confirmed to mask the residue corresponding to Lys445B from interaction, or contact, with a second bevacizumab molecule in a 3D aggregation model of bevacizumab, and crystallographic symmetries.

According to one aspect, a method of identifying a modulator compound having activity for modulating antibody aggregation may comprise generating a 3D aggregation model of two intact bevacizumab molecules, based on the 3D model structure of the intact antibody bevacizumab.

According to one aspect, a method of identifying a modulator compound having activity for modulating antibody aggregation may comprise a step of computer-assisted docking of a compound, to be identified as a compound which interacts preferentially with the residue corresponding to Lys445B, onto the surface of an intact antibody bevacizumab in a 3D monomer model of an intact bevacizumab molecule; and confirming, by visual inspection, that said compound masks the residue corresponding to Lys445B from interaction, or contact, with a second bevacizumab molecule in the 3D aggregation model.

According to another aspect of the invention, there is provided a method of identifying a modulator compound having activity for modulating antibody aggregation comprising: generating a 3D monomer model of the structure of the intact antibody such as bevacizumab as taught herein, or obtained by structural analysis of the intact antibody molecule such as using X-ray crystallography, NMR spectroscopy, or dual polarisation interferometry; performing a computer-assisted docking of a candidate compound onto the surface of the intact antibody; identifying a modulator compound that interacts preferentially, for example as determined by using the interfering volume as described in Example 2, with the lysine residue located in position number 8 of an amino acid sequence having the sequence of SEQ ID NO: 2 comprised in the CH domain of the Fc region of the intact antibody, for example with the lysine residue located in position number 8 of an amino acid sequence having the sequence of SEQ ID NO: 3 comprised in the CH domain of the Fc region of the intact antibody, in particular the lysine residue located in position number 28 of an amino acid sequence having the sequence of SEQ ID NO: 4 comprised in the CH domain of the Fc region of the intact antibody or the lysine residue located in position number 75 of an amino acid sequence having the sequence of SEQ ID NO: 1 comprised in the CH domain of the Fc region of the intact antibody.

Optionally, a compound that has been identified as a compound which interacts preferentially with lysine residue mentioned above on the surface of the intact antibody, may be visually confirmed to mask the said lysine residue from interaction, or contact, with a second intact antibody molecule in a 3D aggregation model of intact antibody as taught by the present description.

According to one aspect, a method of identifying a modulator compound having activity for modulating antibody aggregation may comprise generating a 3D aggregation model of two intact antibody molecules, based on the 3D model structure of the intact antibody and crystallographic symmetries as taught by the present description.

According to one aspect, a method of identifying a modulator compound having activity for modulating antibody aggregation may comprise a step of computer-assisted docking of a compound, to be identified as a compound which interacts preferentially with the lysine residue mentioned herein, onto the surface of an intact antibody in a 3D monomer model of an intact antibody molecule; and confirming, by visual inspection, that said compound masks the said lysine residue from interaction, or contact, with a second antibody molecule in the 3D aggregation model as taught by the present description.

A pre-selection of compounds from a compound library may optionally be carried out, for example, based on the presence of at least one negatively charged anchor group for binding with the antibody, e.g. molecules terminating in a phosphate or phosphonate group, and/or based on the volume of the compound, e.g. molecules having a dimension in the range from 8 to 13 Å.

According to one aspect there is provided a stable antibody formulation comprising an intact antibody, a liquid carrier and a compound obtained according to the above-mentioned method.

According to one aspect of the invention, there is provided a stable antibody formulation comprising a liquid carrier, an intact antibody and a compound of the formula (I):

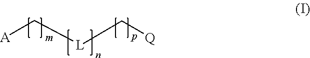

wherein n=0 or 1, m and p are each independently 0 or 1; A is a negatively charged anchor moiety; L is an optional linker group, wherein, when present, L is a C₁-C₆ alkyl, C₁-C₆ carbonyl, C₁-C₆ ether, optionally substituted by one or more group(s) independently selected from a C₁-C₆ alkyl, hydroxy, C₁-C₆ alkoxy, ketone, halo or carboxy group, or a substituted 5- or 6-membered alicyclic, heteroalicyclic, aromatic or heteroaromatic group containing from 0 to 3 heteroatoms selected from a N, O and S, optionally further substituted by one or more group(s) independently selected from a C₁-C₆ alkyl, hydroxy, C₁-C₆ alkoxy, ketone, halo or carboxy group; Q is a cyclic moiety selected from an optionally substituted alicyclic, heteroalicyclic, aromatic or heteroaromatic moiety group comprising 1 isolated to 5 five- or six-membered rings, which may be fused, spiro, or bridged, and 0 to 5 heteroatoms selected from a N, O and S, optionally further substituted by one or more group(s) independently selected from a C₁-C₆ alkyl, hydroxy, C₁-C₆ alkoxy, ketone, aldehyde, carboxy, amine, nitro, thio or halo group, or a pharmaceutically acceptable salt or a tautomer thereof.

According to one aspect, corticosteroids, and specifically betamethasone phosphate and dexamethasone phosphate, are excluded.

Anchor moiety A may preferably be selected from a carboxy, phosphate, phosphonate phosphinate, phosphorothioate, sulfate, sulfonate group, or bioisosteres thereof. Preferably, the anchor moiety A is a phosphonate or a phosphate group.

Mono-, di- and tri-phosphate groups are envisaged. However tri-phosphate groups are less preferred since the many degrees of freedom in the docking of tri-phosphate compounds at the antibody surface tend to lead to a reduction in the number of docking and interfering poses of the molecule successful in interfering with the antibody-antibody aggregation interface. Mono- and di-phosphate groups may be preferred. A mono-phosphate or mono-phosphonate group is preferred as the anchor moiety A.

According to certain embodiments, the linker group L is a substituted tetrahydrofuran group. Where L is a substituted tetrahydrofuran group, substituting groups are preferably independently selected from a hydroxyl or C₁ to C₆ alkoxy.

According to one embodiment, n, m and p are 0.

According to another embodiment, n and m are 1 and p is 0.

The cyclic group Q may preferably be selected from an isolated alicyclic, heteroalicyclic, aromatic or heteroaromatic 6-membered ring, optionally containing 1 or 2 heteroatoms selected from a N, O or S, or an optionally substituted alicyclic, heteroalicyclic, aromatic or heteroaromatic moiety having two five- or six-membered rings, which rings may be fused, and optionally comprising 1 to 5 heteroatoms selected from a N, O or S; optionally substituted by one or more group(s) independently selected from a C₁-C₆ alkyl, hydroxy, C₁-C₆ alkoxy, ketone, aldehyde, carboxy, amine, nitro or halo group. According to another embodiment, the cyclic group Q may be selected from an optionally substituted isolated alicyclic, heteroalicyclic, aromatic or heteroaromatic 6-membered ring, optionally containing 1 or 2 heteroatoms selected from a N, O or S and an optionally substituted alicyclic, heteroalicyclic, aromatic or heteroaromatic moiety having two five- or six-membered rings, which rings may be bridged (e.g. typically via a link selected from —O— and alkoxy (such as optionally substituted methoxy e.g. a O—CH₂ bridge), and optionally comprising 1 to 5 heteroatoms selected from a N, O or S; those rings being optionally further substituted by one or more group(s) independently selected from a C₁-C₆ alkyl, hydroxy, C₁-C₆ alkoxy, ketone, aldehyde, carboxy, amine, nitro or halo group.

According to a preferred embodiment, the cyclic group Q is an optionally substituted pyridine or purine. The purine group Q may optionally be substituted by one or more group(s), e.g. one to three groups, independently selected from amine, halo, hydroxy or C₁-C₆ alkoxy groups. According to a preferred embodiment, Q is a nucleobase, selected from an adenine, guanine, thymine, uracil, xanthine, ethanoadenine, inosine, orotidine, or cytosine.

According to another embodiment, the cyclic group Q may be selected from an optionally substituted isolated heteroalicyclic optionally containing 1 or 2 heteroatoms selected from a N, O or S and an optionally substituted alicyclic, heteroalicyclic, aromatic or heteroaromatic moiety having two five- or six-membered rings, which rings are bridged via an oxygen atom, and optionally comprising 1 to 5 heteroatoms selected from a N, O or S; those rings being optionally further substituted by one or more group(s) independently selected from a C₁-C₆ alkyl, hydroxy, C₁-C₆ alkoxy, ketone, aldehyde, carboxy, amine, nitro or halo group. In a particular embodiment, Q is a monosaccharide or a disaccharide.

According to another preferred embodiment, no linker group L is present and Q is a monosaccharide or a disaccharide. Suitable monosaccharides include glucose, fructose, fucose, galactose, preferred is galactose. Examples of suitable disaccharides include lactose, maltose, sucrose, lactulose, trehalose and cellobiose. In a preferred embodiment, the disaccharide is selected from a lactose, maltose or sucrose.

According to one preferred embodiment, the compound of formula (I) is selected from a monosaccharide phosphate or a disaccharide phosphate. According to a preferred embodiment, the compound of formula (I) is selected from α-D-galactose-1-phosphate, α-lactose-1-phosphate, α-D(+) maltose-1-phosphate and sucrose phosphate, or a pharmaceutically acceptable salt thereof.

According to another aspect there is provided a stable antibody formulation comprising a liquid carrier, an intact antibody and a compound of the formula (A):

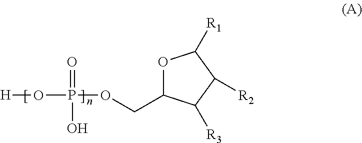

wherein R₁ is a nucleobase selected from the group consisting of adenine, guanine, thymine, uracil, xanthine, ethanoadenine, inosine, orotidine, or cytosine; R₂ is H or OR₄ wherein R₄ is H or a C₁₋₄ alkyl group; R₃ is H or OR₅ wherein R₅ is H or a C₁₋₄ alkyl group; and n is an integral from 1-3, or a pharmaceutically acceptable salt or a tautomer thereof.

According to one embodiment, R₂ and R₃ are each independently H or OH. According to a particular embodiment, R₂ is H and R₃ is OH. According to a particular, embodiment, R₂ and R₃ are both OH.

Particular compounds according to formula (A) include: adenosine 5′-mono-, -di-, or -triphosphate, guanosine 5′-mono-, -di-, or -triphosphate, uridine 5′-mono-, -di-, or -tri-phosphate; cytidine 5′-mono-, -di-, or -triphosphate, deoxyadenosine 5′-mono-, -di-, or -triphosphate, deoxyguanosine 5′-mono-, -di-, or -triphosphate, thymidine 5′-mono-, -di-, or -triphosphate, deoxyuridine 5′-mono-, -di-, or -triphosphate, deoxycytidine 5′-mono-, -di-, or -triphosphate, xanthine 5′-mono-, -di-, or -triphosphate, ethoadenosine 5′-mono-, -di-, or -triphosphate, inosine 5′-mono-, -di-, or -triphosphate, orotidine 5′-mono-, -di-, or -triphosphate. Preferred compounds of formula (A) include adenosine 5′-monophosphate (AMP) and adenosine 5′-diphosphate (ADP), particularly adenosine 5′-monophosphate (AMP).

According to another embodiment, of the invention, the compound of formula (I) is selected from Fludarabine, Tenofovir, Cidofovir, Tiludronate, or pyridoxal phosphate.

According to another embodiment, of the invention, the compound of formula (I) is selected from Fludarabine, Tenofovir, Cidofovir, or Tiludronate.

The compound of formula (I) may be in the form of its free acid, or may be in the form of a pharmaceutically acceptable salt, for example in the form a sodium, potassium or calcium salt, preferably as a mono- or di-sodium salt or of a tautomer.

Compounds of formula (I) may be prepared, or isolated, according to conventional processes known in the art.

Formulations according to the invention may contain one or more compound(s) of formula (I), or a pharmaceutically acceptable salt(s) thereof.

Advantageously liquid preparations of intact antibodies, in particular intact monoclonal antibodies, may be effectively stabilized by the addition of a compound of formula (I) according to the invention.

Compounds of formula (I) have been shown to exhibit surprisingly favourable intermolecular interaction scores on systematic docking with the intact monoclonal antibody bevacizumab such as described in Example 1. Compounds of formula (I) have been shown by computer-assisted systematic docking studies to exhibit preferential binding around the lysine residue corresponding to Lys445B on the Fc region of bevacizumab.

Compounds of the formula (I) can advantageously reduce the propensity of intact antibodies, such as, for example, the intact monoclonal antibody bevacizumab, to form aggregates in liquid formulations.

Formulations, in particular aqueous formulations, of intact antibodies containing a compound of formula (I) according to the invention may exhibit, for example, a between 10 to 80%, e.g. between 30% to 70%, lower proportion of antibody in aggregate form after storage under accelerated storage conditions (e.g. at storage at 40° C.) for between 1 to 30 days, compared to a corresponding formulation of the intact antibody not containing the compound of formula (I).

The present invention allows the preparation of formulations of intact antibody in aqueous carrier wherein less than 20%, even less than 15%, even less than 10% of the antibody is in aggregate form, as determined by MALS coupled to AFFF, during storage at 40° C. for 35 days.

According to one embodiment, the invention provides a formulation according to the invention wherein less than 10% of bevacizumab is in aggregated form as determined by MALS coupled to AFFF during storage at 40° C. for 35 days.

Compounds of the formula (I) have been shown to advantageously induce the reversion, or breaking, of already formed aggregates of intact antibodies, such as, for example, bevacizumab, into an essentially monomeric state.

For example, the addition of a compound of formula (I) to a formulation, in particular an aqueous formulation, of intact antibodies containing already formed aggregates, for instance in which a proportion of at least 20% of the antibody molecules in the formulation are in aggregate form, makes it possible to induce the reversion of a significant proportion of the formed aggregates into an essentially monomeric state. For instance, an increase in the amount of antibody monomers in the formulation of, for example, from 5% to 50%, e.g. from 10% to 30%, may be observed, after addition of a compound of formula (I) according to the invention.

Further, advantageously, compounds of formula (I) according to the invention can provide stabilizing effects on liquid preparations of intact antibodies even when present at very low concentrations.

Advantageously stabilized formulations of intact antibodies, such as bevacizumab, according to the invention, have been shown to have a decreased propensity to aggregate compared to known formulations.

Based on findings of the inventors, it is considered that the efficacy of compounds of formula (I) for reducing the propensity of intact antibodies to form aggregates, and for inducing reversion of already formed aggregates of intact antibody molecules into an essentially monomeric state are due to the interaction of compounds of formula (I) with the residue corresponding to Lys445B on the Fc region of the antibody, thereby interfering, or blocking, the aggregation-inducing contact with the Fab region of a second antibody molecule (as depicted in the aggregation model in FIG. 2A). Thereby the compound of formula (I) inhibits the formation of aggregates between the antibody molecules, due to a mechanism of competitive binding at the aggregation binding site on the antibody molecule.

The formulations of the invention comprise at least one intact antibody. Generally, the formulation of the invention will contain one type of intact antibody, in a native from or in a form conjugated to an accessory molecule. However, the formulations of the invention may comprise more than one intact antibody, e.g. two or three different intact antibodies.

The intact antibody according to the invention is preferably an intact monoclonal antibody. The intact monoclonal antibody may be an immunoglobulin, for example particularly an IgG1, IgG2, IgG2b, IgG3, or IgG4. The intact monoclonal antibody may alternatively be any known therapeutic, diagnostic or preventative intact monoclonal antibody drug, such as, for example Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab or Ustekinumab.

Intact monoclonal antibodies of particular interest include IgG1, IgG4 and monoclonal antibodies having an Fc region substantially similar to that of IgG1, including, for example, Adalimumab, Alemtuzumab, Bapineuzumab, Basiliximab, Bevacizumab, Belimumab, Canakinumab, Cetuximab, Daclizumab, Denosumab, Eculizumab, Efalizumab, Epratuzumab, Figitumumab, Gemtuzumab, Golimumab, Infliximab, Ipilimumab, Motavizumab, Natalizumab, Nimotuzumab, Ocrelizumab, Ofatumumab, Omalizumab, Otelixizumab, Palivizumab, Panitumumab, Pertuzumab, Raxibacumab, Resilizumab, Rituximab, Tocilizumab, Trastuzumab or Ustekinumab.

According to a preferred embodiment, there is provided a stable antibody formulation according to the invention wherein the intact antibody is bevacizumab.

A particular advantage of the use of the monosaccharide phosphate or disaccharide phosphates like α-D-galactose-1-phosphate, α-lactose-1-phosphate, α-D (+) maltose-1-phosphate or sucrose phosphate is that the sugars galactose, lactose, maltose and sucrose are widely found in common foodstuff and are accepted globally for use as food additives. AMP has also the advantage of being widely accepted and used as food additive. AMP is approved by the FDA under GRAS (Generally Recognised As Safe) notification GRN No. 144. AMP is widely used as a flavour enhancer and/or flavour modifier, for example in chewing gum, coffee, tea, sugar substitutes, snack foods, soups and soup mixes. A particular advantage of the sugar phosphates and AMP is that the sugars and AMP are widely commercially available, and at a low cost.

The use of a non-therapeutic compound, e.g. a known excipient or additive compound, such as sugars or AMP as stabilizing agents for liquid formulations of intact antibody presents also further advantages with respect to avoiding potential problems of combinations of the antibody with another therapeutic agent or physiologically active agent as stabilizer, such as problems of reduced antibody activity or even possible undesired side effects or toxicological effects related to the active agent combination. Adenosine phosphates, in particular AMP, have been shown to exhibit stabilizing effects on liquid preparations of intact antibodies, such as for example bevacizumab. AMP has been shown to significantly reduce the propensity of intact antibodies, such as, for example, the intact monoclonal antibody bevacizumab, to form aggregates in liquid formulations. Further, AMP has been shown to induce significant reversion, or breaking, of already formed aggregates of intact antibodies, such as for example bevacizumab, into an essentially monomeric state.

For example, addition of AMP to a liquid formulation of intact monoclonal antibody, such as bevacizumab, containing already formed antibody aggregates has been shown to provide a decrease in the amount of aggregates in the liquid formulation, and an increase in the amount of antibody monomers in the liquid formulation, for instance an increase in the proportion of the antibody present in the monomer form of generally from 10% to 30% may be observed.

AMP has been shown to reduce the propensity of intact monoclonal antibodies, such as bevacizumab, to form aggregates in liquid formulations upon storage. Advantageously, aqueous formulations of intact antibody according to the invention comprising AMP may contain less than 20%, even less than 15%, even less than 10% of the antibody in aggregate form, as determined by MALS coupled to AFFF, on storage at 40° C. for 35 days.

Suitable liquid carriers for the antibody formulation according to the invention include, for example, water, ethanol, polyols, e.g. glycerol, propolylene glycol, polyethylene glycol, vegetable oils, etc. Aqueous carriers may be preferred. Preferred pharmaceutically acceptable carriers include sterile aqueous solutions or dispersions, particularly sterile injectable solutions or dispersions. Injectable solutions or dispersions may typically be based upon injectable sterile saline or phosphate-buffered saline (PBS) or other injectable carriers known in the art.

Aqueous formulations according to the invention may generally have a pH in the range from pH 4.0 to pH 8.0, for example a physiological pH, for example a pH around pH 7.0.

According to the invention there is provided a formulation according to the invention wherein the formulation is a pharmaceutical formulation, notably formulated for administration in a mammal, typically a human mammal.

Pharmaceutical formulations according to the invention may additionally contain pharmaceutically acceptable buffers (e.g. PBS buffer). Pharmaceutical formulations according to the invention may additionally contain pharmaceutically acceptable excipients, such as for example known pharmaceutically acceptable preservatives, antibacterial agents, dispersing agents, suspending agents, wetting agents, emulsifying agents, flavouring agents, colouring agents, etc. Suspending agent include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia.

The desired concentration of intact antibody in the formulation according to the invention, will depend, amongst others, on the particular antibody used, the pathology to be treated, the dosage form, the dosage regime, the patient to be treated, etc. In general, in aqueous formulations of antibody for parenteral administration (e.g. by injection or infusion) concentration of antibody in the range from about 1 mg/ml to about 25 mg/ml, e.g. from about 2 mg/ml to about 20 mg/ml are usual. According to one embodiment, the invention provides a formulation according to the invention wherein bevacizumab is at a concentration in the range from about 1 mg/ml to about 25 mg/ml, preferably from about 2 mg/ml to about 20 mg/ml.

The desired concentration of a compound(s) of formula (I) in the formulation according to the invention will depend, amongst others, on the concentration of the antibody in the formulation, the extent of stabilization desired, etc. For instance, in an aqueous formulations of antibody according to the invention for parenteral administration (e.g. by injection or infusion) a concentration of compound of formula (I) in the range from about 0.01 mg/ml to about 50 mg/ml, e.g. from about 0.1 to about 20 mg/ml, may be envisaged.

Generally the molar ratio of the compound of formula (I) to the intact antibody is in the range from about 0.1:1 to about 500:1, preferably from about 1:1 to about 200:1. In a particular embodiment, the molar ratio of the compound of formula (I) to the intact antibody is in the range from about 1:1 to about 100:1, in particular 1:1 to about 50:1, such as for example from about 1:1 to about 10:1.

Formulations of this invention may be administered in any manner including parenterally, transdermally, rectally, transmucosally, intra-ocular or combinations thereof. Parenteral administration includes, but is not limited to, intravenous (i.v.), intraarterial, intraperitoneal, subcutaneous, intramuscular, intrathecal, and intraarticular. The compositions of the invention may also be administered in the form of an implant, which allows a slow release of the compositions as well as a slow controlled i.v. infusion.

Intraocular administration includes, but is not limited to, injection into the vitreous humour, subconjunctival, subtenon, topical applications. The formulations of this invention may also be administered in the form of an ocular implant, which allows slow release of the compositions.

According to a preferred embodiment, the invention provides a formulation according to the invention wherein the formulation is a pharmaceutical formulation suitable for injection in human (e.g. intravitreal or intravenous). In a particular embodiment, the formulation is a pharmaceutical formulation suitable for ocular injection in human (e.g. intravitreal). In another embodiment, the formulation is a pharmaceutical formulation suitable for intravenous injection in human.

Formulations of the invention, together with a conventionally employed adjuvant, carrier, diluent or excipient may be placed into the form of pharmaceutical compositions and unit dosages thereof, and in such form may be employed as liquids such as solutions, suspensions, emulsions, elixirs, or capsules filled with the same, or in the form of sterile injectable solutions for ocular (including intravitreal cavity) use. Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethyl cellulose, carboxymethyl cellulose, aluminum stearate gel, and hydrogenated edible fats. Emulsifying agents include, but are not limited to, lecithin, sorbitan monooleate, and acacia. Injectable compositions are typically based upon injectable sterile saline or phosphate-buffered saline or other injectable carriers known in the art.

The formulations of the present invention may be provided in the form of a kit comprising in one or more container(s) a formulation according to the invention together with instructions for use of said formulation.

The formulation may be adapted for delivery by repeated administration.

Stabilized intact antibodies according to the invention and formulations thereof, obtainable by a process or a method according to the invention, are useful in the prevention and/or treatment of a disease or a disorder such as immunological diseases, autoimmune diseases, graft rejection, infectious diseases, inflammatory diseases, neurological diseases, neovascular diseases, or oncological diseases.

According to one embodiment, there is provided a formulation according to the invention for use as a medicament.

In particular, formulations according the invention may be envisaged for the prevention or treatment of a disease or a disorder selected from immunological diseases, autoimmune diseases, infectious diseases, inflammatory diseases, neurological diseases, neovascular diseases, or oncological diseases.

According to a particular embodiment of the invention, there is provided a formulation according the invention for the prevention or treatment of a disease or a disorder selected from a cancer, or a neovascular age-related macular degeneration disease (AMD).

According to one embodiment of the invention, there is provided a method of preventing, treating or ameliorating a disease or a disorder selected from immunological diseases, autoimmune diseases, infectious diseases, inflammatory diseases, neurological diseases, neovascular diseases, or oncological diseases, said method comprising administering in a patient in need thereof a prophylactic or therapeutically effective amount of a stable intact antibody formulation according to the invention or a formulation of a stabilized intact antibody obtainable by a process or a method according to the invention.

According to a particular embodiment of the invention, there is provided a method of preventing, treating or ameliorating a neovascular age-related macular degeneration disease (AMD), said method comprising administering in a subject in need thereof a prophylactic or therapeutically effective amount of a stable bevacizumab formulation or a formulation of a stabilized bevacizumab obtainable by a process or a method according to the invention.

According to one aspect, the invention provides a method of preventing, treating or ameliorating a cancer, said method comprising administering in a subject in need thereof a prophylactic or therapeutically effective amount of a stabilized antibody formulation or a formulation of a stabilized bevacizumab according to the invention. Particularly considered cancers include metastatic cancers, e.g. selected from colon or rectal cancer.

Typically, for cancer treatments such as colorectal cancer, the therapeutically effective dose of a stabilized bevacizumab according to the invention is from about 3 mg/kg body weight to about 20 mg/kg body weight.

The dosage administered, as single or multiple doses, to an individual will vary depending upon a variety of factors, including pharmacokinetic properties, patient conditions and characteristics (gender, age, body weight, health, and size), extent of symptoms, concurrent treatments, frequency of treatment and the effect desired.

According to another aspect of the invention, there is provided a method of stabilizing an intact antibody in aqueous solution by combining said intact antibody with a compound of formula (I).

According to one embodiment, there is provided a process for the preparation of an intact antibody or a formulation thereof comprising the steps of:
(i) Combining said intact antibody with a compound of formula (I) into a liquid mixture or forming said intact antibody in a liquid medium containing a compound of formula (I);
(ii) collecting the liquid mixture or liquid medium obtained under step (i) containing the stabilized intact antibody wherein the percentage of monomers of intact antibody is increased as compared to intact antibody prepared in absence of the said compound of formula (I).

Typically, the percentage of monomers of stabilized intact antibody is of about at least 90% after 35 days at 40° C. at 25 mg/ml.

In a particular embodiment, there is provided a method according to the invention wherein the said intact antibody is bevacizumab. For example, bevacizumab used in a method or process according to the invention may be obtained by a process as described in Presta et al., 1997, above.

In a particular embodiment, there is provided a method or process according to the invention wherein the said compound of formula (I) is AMP or ADP, particularly AMP.

In a particular embodiment, there is provided a method or process according to the invention wherein the said compound of formula (I) is GMP.

In a particular embodiment, there is provided a method or process according to the invention wherein the said compound of formula (I) is ATP.

In a particular embodiment, there is provided a method or process according to the invention wherein the said compound of formula (I) is sucrose phosphate.

The method or process according to the invention may also usefully be applied for decreasing the aggregation ability of an intact antibody during its production process and/or for recovering production batches containing already aggregated antibodies by reverting them into an essentially monomeric state.

The method or process according to the invention may be usefully applied for preparing stable formulations of intact antibodies presenting an increased shelf-life and enabling multiple dosing conditioning.

The invention is further illustrated by the following non-limiting examples.

### EXAMPLES

The following abbreviations refer respectively to the definitions below:

mM (millimolar), nm (nanometers), AFFF (asymmetrical flow field-flow fractionation), MALS (multi-angle light scattering), UV (ultraviolet).

### Example 1

### Determination of the 3D Aggregation Model of Bevacizumab

To build the 3D model of bevacizumab, the Protein Data Bank crystal structures of (i) the 2 Fab regions of bevacizumab in complex with VEGF (PDB identity: 1BJ1, 2.40 Å resolution, space group P2₁) (Muller et al., 1998, Structure, 6:1153-1167) and (ii) the full length mouse IgG1 containing the IgG1 Fab and Fc regions (PDB identity: 1IGY, 3.2 Å resolution, space group P2₁) (Harris et al., 1998, above) were used.

The initial 3D model of bevacizumab resulted from the following modelling steps that were carried out in Sybyl 8.0 (Tripos Inc.):

1) Structural superposition of the corresponding Fabs of bevacizumab and IgG1 according to sequence alignments, and
2) Replacement of mouse IgG1 Fabs by the Fabs of bevacizumab and connection of the Fabs of bevacizumab with the hinge-Fc of mouse IgG1.

Further, a refined model was generated where the hinge-Fc region of mouse IgG1 was “humanized” by “mutating” residues of this region to match the sequence of the hinge-Fc region from the only sequence of human IgG1 available with a corresponding suitable crystal structure (PDB identity: 1 HZH) (Saphire et al., 2001, above). The full human IgG1 crystal structure (PDB identity: 1 HZH) could not be used due to its unusual crystal symmetry (strong distortion of the orientation of the Fabs with respect to the axis of the Fc region). Similar results were obtained with the initial model (non-humanized) and the refined model (humanized) supporting that this model is relevant for intact therapeutic antibodies having a human Fc region.

The connecting region of the initial model was submitted to energy minimization using Sybyl 8.0 default parameters and keeping the disulfide bridges intact. The quality of the resulting model was assessed using Procheck (Laskowski et al., 1993, J. Appl. Cryst., 26, 283-291) Upon Ramachandran plot analysis (in Procheck) of the amino acid conformations using a resolution mean between the crystal structures of both the Fc and the Fabs, critical side chains were corrected for distortion in Sybyl and the procedure repeated until reaching conformations comparable to the input crystal structures. The resultant 3D model of bevacizumab is depicted in FIG. 1.

To obtain the aggregation model of bevacizumab, first, the crystallographic symmetry (P2₁) of the full IgG1 crystal structure was built, and then the obtained layer was translated along the unit cell, using Deep View Swiss PDB viewer (Guex and Peitsch, 1997, Electrophoresis, 18, 2714-2723). After having visually inspected all the translations obtained, only the translation having close crystal contacts, i.e. in the order of 4 Å (corresponding to the first translation), was saved in Protein Data Bank (pdb) format. The 3D bevacizumab structure model was overlaid upon it according to the carbon α positions in Sybyl. Upon displaying the bevacizumab 3D structure model and its translation, one notes that the bottom of one Fc comes to lie in between the two Fab regions of the second bevacizumab. The two Fc regions of the two antibodies would be located at the same plane if the first Fc would not be slightly turned away from this ideal case so as to interact with one Fab from the other antibody. The resultant aggregation model of bevacizumab is depicted in FIGS. 2A to 2B.

### Example 2

### Molecular Interaction Study of Compounds of Formula (I) with the Surface of the Antibody Bevacizumab

Small molecule setup for docking was done in Sybyl 8.0. Hydrogens were added to the small molecular weight molecules, each phosphate was left unprotonated and Gasteiger-Huckel charges were added. The resulting molecules were minimized using 100 steps of the default Powell minimization protocol of Sybyl 8.0. Systematic docking was then performed with FlexX 3.1.3 all over the bevacizumab antibody surface (Fabs and Fc), divided into several segments of 10 Å around each arginine and lysine. 10 poses per molecule and docking site were generated. The docking poses were scored with the FlexX scoring function and evaluated by analyzing the attributed bevacizumab-small molecule interaction score, visually inspecting all poses and retaining the ones sticking out of the bevacizumab surface (i.e. inside of a volume of interference as defined below and in FIG. 7). The number of docking poses successful in interfering with the bevacizumab-bevacizumab interaction interface (out of 10 solutions) was a key selection/analysis criterion for the small molecular weight compounds.

A volume of interference including the breaking poses is defined as a cylinder as represented in FIG. 7, having the centre of its base defined to be the Cα atom of the lysine residue corresponding to Lys445, with the plane of the base including the N atom of the lysine residue corresponding to Lys445, and the radius was set to 7 Å. A height of 12 to 15 Å was drawn orthogonally from the base using Fc atoms situated approximately on the surface of the circle to the adjacent Fab of the other antibody monomer. According to this representation, it can be seen that AMP and triamcinolone acetonide phosphate (TAP), a non-breaker used as negative control, overlap in vicinity to the Fc, with their phosphates both interacting with the lysine residue corresponding to Lys 445. However, while AMP is occupying sufficient volume and space to interfere with the Fab of the adjacent antibody, TAP is generally not. An interference scoring from 0 to 5 (a scoring from 0 to 2, defining an absence of or marginal aggregation breaking propensity, and 3 to 5, defining significant aggregation breaking propensity) can be defined for modulator candidates as described in Table 2 below:

TABLE 2

| Interfering | |
|---|---|
| score | Associated criteria |
| 5 | Majority of poses interfering, all poses completely inside |
| | the cylinder and root mean square deviation |
| | of heavy atoms ≦2 Å |
| 4 | Majority of poses interfering, majority of poses completely |
| | inside the cylinder |
| 3 | Majority of poses interfering, majority of poses not |
| | completely inside the cylinder |
| 2 | 3/10 poses interfering or 3/10 poses completely inside the |
| | cylinder |
| 1 | 2/10 poses interfering or 2/10 poses completely inside the |
| | cylinder |
| 0 | None of the poses either interfering nor inside the cylinder |

The scores obtained for the 10 best FlexX-scored docking poses out of 100 are listed in Table 3 below for several aggregation breakers and non-breaking molecules as controls (Triamcinolone acetonide (TA) and triamcinolone acetonide phosphate (TAP)).

TABLE 3

| | Interfering |
|---|---|
| Molecule | Score |
| AMP | 5 |
| ADP | 3 |
| ATP | 2 |
| Sucrose phosphate | 5 |
| Cidofovir | 3 |
| Tenofovir | 3 |
| Tiludronate | 3 |
| Amifostine | 3 |
| Fludarabine | 3 |
| TA | 0 |
| TAP | 2 |

Results showed that compounds of formula (I), for example AMP, α-lactose-1-phosphate, α-D(+)mannose-1-phosphate, Fludarabine, Tenofovir, Cidofofir and Tiludronate, docked to the lysine residue corresponding to Lys445B effectively and were positioned in such a way as to interfere with the adjacent antibody.

As seen in Table 3, both AMP and sucrose phosphate indicate strong interfering poses among the modelled population. A decrease in the interfering score from AMP to ADP and ATP is consistent with what was expected, as every phosphate group adds substantial degrees of freedom that make it more difficult for the docking program to find similar poses in terms of root mean square deviations (RMSDs), i.e. RMSDs ≦2 Å. Experiments have confirmed that ATP is a less strong breaker than AMP.

Cidofovir, tenofovir, tiludronate, amifostine and fludarabine are predicted by this scoring scale to have intermediate aggregation breaking properties, probably in the same range as ADP.

### Example 3

### Comparison of the Stability of Bevacizumab Alone and in Association with Adenosine 5′-Monophosphate (AMP)

Four different samples were tested:

A commercial formulation of bevacizumab (Avastin®, Roche Pharma, Reinach, Switzerland) comprising 25 mg/mL bevacizumab in 51 nM phosphate buffer, pH 6.2 containing 60 mg/mL trehalose dehydrate and 0.04% polysorbate 20) was dialyzed overnight into isotonic buffers to reduce excipients present in the commercial product and to change the pH. A 50 mM phosphate buffer pH 7.0 was used. The buffer choice was based on a pH range and buffer capacity that is physiologically tolerated and that is acceptable for the stability of antibodies.

After dialysis, the bevacizumab preparation with a concentration of 25 mg/mL was stored for 7 days at a temperature of 40° C. at pH 7.0 to stress the antibody and induce the formation of aggregates.

A first sample of bevacizumab was separated (in order to test aggregation of bevacizumab alone).

Adenosine 5′-monophosphate powder (purity 99%, Acros Organics) was added in three different concentrations, to the stressed bevacizumab obtaining the following molar ratios:
i. bevacizumab: AMP 1:153
ii. bevacizumab: AMP 1:15.3
iii. bevacizumab: AMP 1:1.53

All samples were stored at 40° C. during 28 days. Samples were analyzed directly after preparation (t₀) and after 7, 14 and 28 days. The aggregation state of the antibodies was measured by multi-angle light scattering (MALS) after separation by asymmetrical flow field-flow fractionation (AFFF). The concentration of bevacizumab was determined by UV spectroscopy at 280 nm, based upon an extinction coefficient of 1.7 cm ml/mg. Data were collected and analysed with the Astra software (Wyatt Technology Europe GmbH, Dernbach, Germany). The aggregation state was expressed as the percentage of monomers versus time.

Further control experiments on the stability of bevacizumab alone were carried out: The concentration effect (5, 10, 18 and 25 mg/ml in 50 mM phosphate buffer pH 6.2) and the effect of pH as well as storage temperature (pH 5.0 and pH 7.0 at 4° C., 25° C. and 40° C. during 35 days) on antibody stability.

The association of bevacizumab with AMP causes a surprising stabilization of the antibody in comparison with the sample of bevacizumab alone (FIG. 3). After 14 days of storage at a temperature of 40° C. at pH 7.0, the percentage of monomers in the formulations of bevacizumab with AMP is higher than 94% (n=3). After 28 days of storage at 40° C. at pH 7, the percentage of monomers is still around 90% (n=3) (FIG. 3) for a molar ratio of bevacizumab:AMP=1:153; and is still higher than 80% after 14 days of storage at 40° C. at pH 7.0 and higher than 76% (n=3) after 28 days of storage at 40° C. at pH 7.0 for a molar ratio of bevacizumab:AMP=1:15.3 or 1:1.53. This is compared to average monomer percentages (n=3) of 75% after 14 days of storage at 40° C. at pH 7.0, and 71% after 28 days of storage at 40° C. at pH 7.0 for bevacizumab alone.

These data clearly show that the combination of an intact antibody such as bevacizumab with a compound of formula (I) such as AMP leads advantageously to stabilized antibody formulations.

### Example 4

### Effect of Adenosine 5′-Monophosphate (AMP) on a Commercial Formulation of Bevacizumab (Avastin®)

Samples of a commercial formulation of bevacizumab (Avastin®, Roche Pharma, Reinach, Switzerland) are combined with AMP at three molar ratios (1:1, 1:10 and 1:100 Avastin®:AMP). All samples are stored at a temperature of 40° C. for 28 days and the stability is measured as described in Example 3 and compared to a sample of Avastin® alone stored under the same conditions.

Compared to the sample of the commercial Avastin® formulation alone, a significant stabilization of the antibody (increase in the amount of monomers) is observed for both the 1:10 and 1:100 samples (p<0.05). For the 1:10 sample, a significant stabilization is observed at t₁, t₁₄ and t₂₈, whereas, for the 1:100 sample, such a stabilization is observed only at longer incubation times (t₁₄ and t₂₈) (FIG. 2). Therefore, compared to the other molar ratios 1:1 and 1:100, the 1:10 sample results in a better stabilization of the antibody. In conclusion, those results confirm those of Example 3 and show that a compound according to formula (I) such as adenosine 5′-monophosphate (AMP) is also able to further stabilize an unmodified commercial antibody formulation.

### Example 5

### Comparison of the Stability of Bevacizumab Alone and in Association with Guanosine 5′-Monophosphate (GMP), Adenosine 5′-Triphosphate (ATP) or Sucrose Phosphate

A commercial formulation of bevacizumab (Avastin®, Roche Pharma, Reinach, Switzerland) was pre-stressed after dialysis into PBS at pH 7.0 as described in Example 3 (for 7 days at a temperature of 40° C.). After pre-stressing, Avastin® samples were combined with either ATP, GMP or sucrose phosphate at three Avastin®: compound of formula (I) molar ratios (1:1, 1:10 and 1:100). All samples are stored at a temperature of 40° C. for 28 days and stability is measured as described in Example 3 and compared to a sample of Avastin® alone stored under the same conditions. For GMP, a dilution of GMP was made in PBS pH 7.0 and pH was adjusted to 7.0 before the combination with Avastin® to prevent the risk of higher order aggregates caused by the addition of NaOH directly to the antibody formulation. For sucrose phosphate, a concentration-dependent stabilization is observed: At all timepoints, the 1:100 formulation is leading to the best stabilization, followed by the 1:10 and thereafter the 1:1 sample.

A concentration dependent stabilization of Avastin® is observed after addition of ATP up to 14 days. At t₂₈, no significant difference is observed between the sample of Avastin® alone and the 1:1 and 1:10 combinations. The 1:100 sample shows a significant stabilization of the antibody after 28 days of storage, although a small percentage of aggregates is also observed. These aggregates are probably due to the adjustment of the pH of this sample. A concentration dependent stabilization of Avastin® is also observed after addition of GMP: At all timepoints, the 1:100 formulation is the most effective in aggregation breaking, followed by the 1:10 and thereafter the 1:1 sample.

Therefore, at an initial stage (e.g. after 1 day of storage at 40° C.: t₁), a stabilizing effect is observed for all three molar ratios (FIG. 3A) after the addition of ATP or sucrose phosphate. GMP seems to be less effective as only the 1:100 sample shows an ability to stabilize the antibody, whereas both the 1:1 and 1:10 samples are destabilizing. At later stage (e.g. 28 days of storage at 40° C.: t₂₈), ATP still shows a significant stabilizing effect on the antibody for the 1:100 samples, however the 1:1 and 1:10 samples show a similar stability as the antibody alone (FIG. 3B). For sucrose phosphate, the concentration-dependent stabilizing effect continues up to 28 days of storage at 40° C. Thus, although ATP shows aggregation breaking effects, these effects are most pronounced directly after addition of the excipient to the antibody. It appears that it takes more time for GMP to interact with the antibody and to interfere with the formation of antibody dimers.

In conclusion, excipients of formula (I) possess stabilizing properties. Short-term effects on the antibody are most pronounced for ATP and sucrose phosphate, whereas GMP shows the most distinct stabilizing properties after 28 days of storage at 40° C.

### Example 6

### Comparison of the Stability of Antibodies Alone and in Association with a Compound of the Invention

Stabilizing effects of compounds of formula (I) according to the invention on various antibodies are assessed as follows:

### Long-Term Stability Studies

The antibody at a concentration of 25 mg/mL in 20 mM histidine buffer pH 6.0 is combined with a compound of formula (I) (such as AMP) from a stock solution in the same buffer, at molar ratios of antibody:compound of 1:1 and 1:10 in the same buffer. The resulting samples where the antibody is at a concentration of 20 mg/ml or higher are then stored either at normal storage temperature (5° C.) or at elevated temperatures (e.g. 25° C. or 40° C.). Aggregation state is then measured during storage such as immediately after sample preparation, 2 weeks, 1 month, 3 months and 6 months after starting storage based on the proportions of monomers, dimers and larger antibody aggregates in each samples by various techniques such as Asymmetrical-Flow Field-Flow-Fractionation (AFFF), Size Exclusion Chromatography, or Analytical Ultracentrifugation. Comparison of aggregation state in the presence and in the absence of compounds of formula (I) demonstrates their ability to prevent aggregation.

### Short-Term Stability Studies Under Stress Conditions

The antibody at a concentration of 25 mg/mL in 20 mM histidine buffer pH 6.0 is pre-stressed using known aggregating conditions (e.g. temperature, pH, agitation for example as described in Kiese et al., 2008, Journal of Pharmaceutical Sciences, 97(10), 4347-4366) followed by the addition of a compounds of formula (I) such as AMP at molar ratios of Mab:compound of 1:1 and 1:10 in buffer. The resulting samples where the antibody is at a concentration of 20 mg/ml or higher are then analyzed for determining their aggregation status immediately after the addition of compounds of formula (I) and 1 week after starting, based on the proportions of monomers, dimers and larger antibody aggregates in each samples by various techniques such as Asymmetrical-Flow Field-Flow-Fractionation (AFFF), Size Exclusion Chromatography, or Analytical Ultracentrifugation. Comparison of aggregation state in the presence and in the absence of compounds of formula (I) demonstrates their ability to reverse aggregation.

1-39. (canceled)

40. A method of stabilizing an intact antibody in a liquid carrier comprising modulating aggregation of said intact antibody by binding to, or masking, a specific lysine in the Fc region of the intact antibody molecule, wherein said lysine residue corresponds to position number 8 of SEQ ID NO: 2 comprised in the Fc region, in particular in a CH domain of the said Fc region of the said intact antibody molecule.

41. The method according to claim 40, wherein said lysine residue is located in position number 8 of an amino acid sequence of SEQ ID NO: 3 comprised in the Fc region, in particular in a CH domain of the said Fc region, of the said intact antibody molecule.

42. The method according to claim 40, wherein said lysine residue is located in position number 28 of an amino acid sequence of SEQ ID NO: 4 or 5 comprised in the Fc region, in particular in a CH domain of the said Fc region of the said intact antibody molecule.

43. The method according to claim 40, wherein said lysine residue is located in position number 75 of SEQ ID NO: 1 comprised in the Fc region, in particular in a CH domain of the said Fc region of the said intact antibody molecule.

44. A stable antibody formulation comprising a liquid carrier, an intact antibody and a modulator compound, said modulator compound having binding affinity for a specific Lysine in the Fc region of the intact antibody molecule, wherein the said lysine residue is in position number eight of an amino acid sequence of SEQ ID NO: 2 comprised in the Fc region, in particular in the CH domain of the said Fc region of the said intact antibody molecule.

45. The stable antibody formulation according to claim 44, wherein the modulator compound has the formula (I): 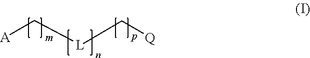
wherein n, m, p, A, L and Q are defined in claim 44, or a pharmaceutically acceptable salt or a tautomer thereof.

46. The stable antibody formulation according to claim 44, wherein the intact antibody is conjugated to an accessory molecule or a native antibody.

47. The stable antibody formulation according to claim 44, wherein the intact antibody is bevacizumab.

48. The stable antibody formulation according to claim 44, wherein the formulation is a pharmaceutical formulation.

49. A stable antibody formulation comprising a liquid carrier, an intact antibody and a compound of the formula (I): 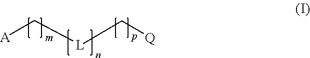
wherein n=0 or 1, m and p are each independently 0 or 1; A is a negatively charged anchor moiety selected from a carboxy, phosphate, phosphonate, phosphinate, phosphorothioate, sulfate, or sulfonate moiety; L is a C₁-C₆ alkyl, C₁-C₆ carbonyl, C₁-C₆ ether, optionally substituted by one or more group(s) independently selected from a C₁-C₆ alkyl, hydroxy, C₁-C₆ alkoxy, ketone, halo or carboxy group, or a substituted 5- or 6-membered alicyclic, heteroalicyclic, aromatic or heteroaromatic group containing from 0 to 3 heteroatoms selected from a N, O or S, optionally further substituted by one or more group(s) independently selected from C₁-C₆ alkyl, hydroxy, C₁-C₆ alkoxy, ketone, halo or carboxy group; Q is a cyclic moiety selected from an optionally substituted alicyclic, heteroalicyclic, aromatic or heteroaromatic moiety group comprising 1 to 5 five- or six-membered rings which may be fused, spiro or bridged, and 0 to 5 heteroatoms selected from a N, O or S optionally further substituted by one or more group(s) independently selected from a C₁-C₆ alkyl, hydroxy, C₁-C₆ alkoxy, ketone, aldehyde, carboxy, amine, nitro, thio or halo group, or a pharmaceutically acceptable salt or a tautomer thereof.

50. The stable antibody formulation according to claim 49, wherein A is selected from a mono-, di- or tri-phosphate group.

51. The stable antibody formulation according to claim 49, wherein Q is selected from an optionally substituted isolated alicyclic, heteroalicyclic, aromatic or heteroaromatic 6-membered ring, optionally containing 1 or 2 heteroatoms selected from a N, O, or S and an optionally substituted alicyclic, heteroalicyclic, aromatic or heteroaromatic moiety having two five- or six-membered rings, which rings are bridged via an oxygen atom, and optionally comprising 1 to 5 heteroatoms selected from a N, O, or S; those rings being optionally further substituted by one or more group(s) independently selected from a C₁-C₆ alkyl, hydroxy, C₁-C₆ alkoxy, ketone, aldehyde, carboxy, amine, nitro or halo group.

52. The stable antibody formulation according to claim 50, wherein m and n are I and p is 0.

53. The stable antibody formulation according to claim 50, wherein m, n, and p are 0.

54. The stable antibody formulation according to claim 49, wherein L is tetrahydrofuran.

55. The stable antibody formulation according to claim 49, wherein the compound of formula (I) is selected from a monosaccharide phosphate or a disaccharide phosphate or a pharmaceutically acceptable salt thereof.

56. The stable antibody formulation according to claim 55, wherein the compound of formula (I) is selected from α-D-galactose-1-phosphate, α-lactose-1-phosphate, α-D(+) maltose-1-phosphate or sucrose phosphate, or a pharmaceutically acceptable salt thereof.

57. The stable antibody formulation according to claim 49, wherein the compound of formula (I) is sucrose phosphate.

58. The stable antibody formulation according to claim 49, wherein the compound of formula (I) is selected from Fludarabine, Tenofovir, Cidofovir or Tiludronate.

59. A pharmaceutical unit dosage form suitable for ocular or intravenous administration to a mammal comprising an antibody formulation according to claim 44 in a suitable container.

60. A method of stabilizing an intact antibody in a liquid carrier by combining said intact antibody with a compound of formula (I); 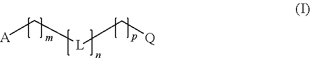
wherein n, m, p, A, L and Q are defined in claim 49, or a pharmaceutically acceptable salt or a tautomer thereof.

61. A process for the preparation of an intact antibody or a formulation thereof comprising the steps of:
(i) combining an intact antibody with a compound of formula (I) wherein n, m, p, A, L and Q are defined in claim 49, or a pharmaceutically acceptable salt thereof, into a liquid mixture or forming said intact antibody in a liquid medium containing a compound of formula (I);
(ii) collecting the liquid mixture or liquid medium obtained under step (i) containing the stabilized intact antibody thereof wherein the percentage of monomers of the intact antibody is increased as compared to an intact antibody prepared in absence of the said compound of formula (I).

62. A method of preventing, treating or ameliorating a disease or a disorder selected from a cancer, rheumatoid arthritis, transplant rejection, blood coagulation, infection with respiratory syncitial virus (RSV), Crohn's disease, cardiovascular disease, auto-immune disease, graft rejection, asthma, paroxysmal nocturnal hemoglobulinuria, psoriasis, or a neovascular age-related macular degeneration disease (AMD), said method comprising administering to a subject in need thereof a prophylactic or therapeutically effective amount of a stable bevacizumab formulation according to claim 47.


SEQUENCE LISTING (8 sequences)

SEQ ID NO: 1 - protein, 107 aa, Homo sapiens
KGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 15 aa, Artificial sequence
  synthetic construct
YSKLXVXKXXWXXXN

SEQ ID NO: 3 - protein, 15 aa, Homo sapiens
YSKLTVDKSRWQQGN

SEQ ID NO: 4 - protein, 60 aa, Homo sapiens
PENNYKTTPPVLDSDGSFFLYSKLTVDKSRWQQGNVFSCSVMHEALHNHYTQKSLSLSPG

SEQ ID NO: 5 - protein, 61 aa, Homo sapiens
PENNYKTTPPVLDSDGSFFLYSKLTVDKSRWQQGNVFSCSVMHEALHNHYTQKSLSLSPGK

SEQ ID NO: 6 - protein, 57 aa, murine
APENYKNTQPIMDTDGSYFVYSKLNVQKSNWEAGNTFTCSVLHEGLHNHHTEKSLSH

SEQ ID NO: 7 - protein, 14 aa, Homo sapiens
PSRDELTKNQVSLT

SEQ ID NO: 8 - protein, 457 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK